(19) United States
(12) Patent Application Publication
(10) Pub. No.: US 2018/0318199 A1
(43) Pub. Date: Nov. 8, 2018

(54) Pharmaceutical Composition Having Anti-Aging Properties against High-Glucose

(71) Applicant: TAIWAN LEADER BIOTECH CORP., Taipei (TW)

(72) Inventors: Chin-Chung Lin, Taipei (TW); Sheng-Yang Wang, Taipei (TW)

(21) Appl. No.: 15/864,373

(22) Filed: Jan. 8, 2018

Related U.S. Application Data
(63) Continuation-in-part of application No. 15/585,270, filed on May 3, 2017, now abandoned.

(51) Int. Cl. A61K 8/63; A61Q 19/08; A61K 8/9728

(52) U.S. Cl. CPC A61K 8/63; A61K 8/9728; A61Q 19/08

(57) ABSTRACT

The present method has anti-aging properties against high-glucose. The present invention reveals the anti-aging properties of antcin M (ANM) and elucidates the molecular mechanism underlying the effects. It is found that exposure of human normal dermal fibroblasts (HNDFs) to high-glucose (HG) for 3 days, cell phase arrest and senescence are accelerated. As confirmed through experiments, co-treatment with ANM significantly attenuates HG-induced growth arrest and promotes cell proliferation. In addition, treatment with ANM eliminates HG-induced reactive oxygen species through the induction of anti-oxidant genes via transcriptional activation of NF-E2 related factor-2 (Nrf2). Treatment with ANM abolishes HG-induced stress-induced premature senescence as evidenced by reduced senescence-associated β-galactosidase activity. Also, the HG-induced decline in aging-related marker protein, senescence marker protein-30, is rescued by ANM. Furthermore, treatment with ANM increases expression of silent mating type information regulation 2 homologs 1 (SIRT-1), and prevents SIRT-1 depletion.

### CROSS-REFERENCE TO RELATED APPLICATIONS

This application is a continuation-in-part of United States Application 15/585,270, filed May 3, 2017 (7000.830), the disclosure of which is incorporated by reference in its entirety.

### TECHNICAL FIELD OF THE INVENTION

The present invention relates to an anti-aging reagent and method; more particularly, to providing antcin M (ANM) to eliminate hyperglycemia-accelerated premature senescence in human normal dermal fibroblasts (HNDFs) by direct activation of NF-E2 related factor-2 (Nrf2) and silent mating type information regulation 2 homologs 1 (SIRT-1).

### DESCRIPTION OF THE RELATED ARTS

Premature skin aging is caused by several factors, including intense physical and psychological stress, alcohol intake, poor nutrition, environmental pollution, UV exposure and diabetes. Hyperglycemia is a characteristic feature of diabetes mellitus (DM), and the clinical involvement of skin in diabetic complications such as impaired wound healing, foot ulceration, and premature skin aging are well studied. The proliferative capacity of skin fibroblasts harvested from diabetic subjects is reduced, as they have reduced replicative life span. Likewise, HNDFs harvested from normal donors cultured in hyperglycemic medium result in reduction in the population doubling required to reach replicative senescence. These findings suggest that accelerated cellular senescence resembling premature aging is also included in complications of diabetes.

Replicative senescence of human diploid fibroblasts (HDFs) or melanocytes is caused by the exhaustion of proliferative potential. Many proliferative cell types such as endothelial cells, lung cells, retinal pigment epithelial cells, melanocytes and skin fibroblasts undergo stress-induced premature senescence (SIPS) in vitro when exposed to sub-cytotoxic concentrations of oxidative-stress stimuli such as hydrogen peroxide (H2O2), hypoxia (pO2), tert-butylhydroperoxide (tert-BHP), ultraviolet radiation (UV) and hyperglycemia. Over the last two decades several studies have been conducted to elucidate the cellular and molecular mechanisms of SIPS in skin fibroblasts, and have identified oxidative stress as playing a crucial role in the development of SIPS. Increasing oxidative stress is frequently associated with aging and age-related disorders. Reactive oxygen species (ROS) act as signaling molecules, whereas increased levels are damaging for DNA, proteins, and lipids as well as detrimental to cellular functions. Under normal physiological conditions, cells are equipped with an anti-oxidant defense system to eliminate pro-oxidants, but this system fails with over production of ROS. Substantial evidence indicates that hyperglycemia- and hydrogen peroxide-induced ROS generation promotes cellular senescence and growth arrest, thus resulting in SIPS in fibroblasts. Accordingly, prevention of hyperglycemia-associated dermal fibroblast senescence may be a potential target to arrest the development of premature skin aging.

Many dietary components exert beneficial effects on the aging process, such as polyphenols, flavonoids, terpenoids, vitamins and omega-3-fatty acids. These components exert anti-oxidant effects not only by scavenging free radicals but also by modulating signal transduction pathways such as de novo expression of antioxidant genes including hemoxygenase-1 (HO-1), NAD(P)H:quinone oxidoreductase-1 (NQO-1), glutathione-S-transferase (GST), γ-glutamylcestine synthetase (γ-GCLC), and superoxide dismutase (SOD). Transcriptional activation of antioxidants or detoxifying genes is predominantly regulated by a redox-sensitive transcription factor NF-E2 related factor-2 (Nrf2). Both in vitro and in vivo studies suggest that dietary phytochemicals are able to activate Nrf2 signaling thereby ameliorating the anti-oxidant defense system.

Accumulating evidence suggests that the activation of silent mating type information regulation 2 homologs (sirtuins), a family of NAD+-dependent class III histone deacetylases, extends life span and promotes longevity and healthy aging. In particular, sirtuin-1 (SIRT-1), a mammalian ortholog of yeast SIRT-2 plays a functional role in human aging by means of deacetylation, a protein activity that plays a crucial role in cellular senescence, such as p53, Forkhead box protein O1 (FoxO1) and E2F1. A previous study demonstrated that hyperphosphorylation of SIRT-1 at serine 47 (S47) by mitogen-activated protein kinases (MAPKs) resulted SIRT-1 depletion and increased cellular senescence.

Antrodia cinnamomea (A. cinnamomea) is a precious medicinal mushroom that has long been used as a traditional Chinese medicine for the treatment of liver diseases, food and drug intoxication, diarrhea, abdominal pain, hypertension, allergies, skin itching and tumorigenic diseases. A. cinnamonea is one of the richest sources of unique compounds such as antcins, anticinates, antrodins and antroquinonls. Recent study has shown that the chemical fingerprints of A. cinnamomea and its relative specie Antrodia salmonea (A. salmonea) are mostly identical; however, a few compounds including ANM and methyl anticinate K (ANK) are only identified in A. salmonea. Antcins, steroid-like compounds, exhibited various biological effects such as anti-oxidant, anti-inflammation, anti-cancer and cardioprotection. Previously, it is reported that antcin C protects human hepatic cells from oxidative injury through the activation of Nrf2-dependent anti-oxidant genes. However, the other effects of these potentially beneficial compounds have not been investigated. Oxidative stress is one of the major factors that plays a key role in the onset of senescence. Hyperglycemia-induced oxidative stress-mediated senescence has been well-studied in human vascular endothelial cells. However, very few studies have investigated this phenomenon in other cell systems and, therefore, it is necessary to establish a human dermal fibroblast senescence model. Hence, the prior arts do not fulfill all users' requests on actual use.

### SUMMARY OF THE INVENTION

The main purpose of the present invention is to protect HNDFs from hyperglycemia-induced cell phase arrest by ANM.

Another purpose of the present invention is to protect HNDFs from hyperglycemia-induced oxidative damage by ANM.

Another purpose of the present invention is to activate Nrf2-mediated anti-oxidant genes in HNDFs by ANM.

Another purpose of the present invention is to increase expression of SIRT-1 in HNDFs by ANM.

Another purpose of the present invention is to protect and extend life span of Caenorhabditis elegans (C. elegans) under stress condition.

To achieve the above purposes, the present invention is a pharmaceutical composition having anti-aging properties against high-glucose (HG), comprising ANM from A. salmonea as an active ingredient, and a pharmaceutically acceptable carrier or excipient, where the pharmaceutical composition protects HNDFs to prevent hyperglycemia-induced cell phase arrest and enhance cell proliferation; the pharmaceutical composition protects HNDFs to prevent hyperglycemia-induced G0/G1 phase arrest and senescence; the pharmaceutical composition inhibits HG-induced reduction in G1-S transition regulatory proteins comprising cyclin D, cyclin E, cyclin-dependent kinase (CDK4), CDK6, CDK2 and protein retinoblastoma (pRb); the pharmaceutical composition protects HNDFs to prevent hyperglycemia-induced oxidative damage; the pharmaceutical composition activates Nrf2-mediated anti-oxidant genes and eliminates HG-induced ROS and the anti-oxidant genes comprises HO-1 and NQO-1; the pharmaceutical composition abolishes SIPS in presence of HG by reducing senescence-associated β-galactosidase (SA-β-gal) activity in HNDFs; the pharmaceutical composition reduces expression of senescence-associated marker proteins in HNDFs, including p21CIP1, p16^{INK4A}, and p53/FoxO1 acetylation; the pharmaceutical composition increases expression of SIRT-1 in HNDFs; the pharmaceutical composition enhances expression of senescence marker protein-30 (SMP30) in HG-induced HNDFs; and the pharmaceutical composition protects and extends life span of C. elegans under stress condition. Accordingly, a novel pharmaceutical composition having anti-aging properties against HG is obtained.

Key words: Antcin M, Antrodia salmonea, hyperglycemia, stress-induced premature senescence, SIRT-1, Nrf2

### BRIEF DESCRIPTION OF THE DRAWINGS

The present invention will be better understood from the following detailed description of the preferred embodiment according to the present invention, taken in conjunction with the accompanying drawings, in which

FIG. 1A˜FIG. 1I are the views showing the stress-induced premature senescence accelerated by high-glucose (HG) in dermal fibroblasts;

FIG. 2A˜FIG. 2D are the views showing the HG-induced senescence affected by the antcins in human normal dermal fibroblasts (HNDFs);

FIG. 3A˜FIG. 3C are the views showing the HG-induced growth arrest blocked by antcin M (ANM) in HNDFs;

FIG. 4A˜FIG. 4H are the views showing the HG-induced senescence inhibited by ANM in HNDFs;

FIG. 5A˜FIG. 5I are the views showing the NF-E2 related factor-2 (Nrf2)-dependent antioxidant genes activated by ANM in HNDFs;

FIG. 6A˜FIG. 6C are the views showing the HG-induced oxidative stress in Nrf2 silenced cells failed to be protected by ANM;

FIG. 7A˜FIG. 7I are the views showing the silent mating type information regulation 2 homologs 1 (SIRT-1) upregulated by ANM in HNDFs;

FIG. 8A˜FIG. 8C are the views showing the life span of wild-type Caenorhabditis elegans (C. elegans) extended by ANM from oxidative stress;

FIG. 9A˜FIG. 9H are the views showing the HG-induced senescence prevented by ANM in human umbilical vein endothelial cells (HUVECs); and

FIG. 10 is the view showing the HNDFs and the HUVECs protected from HG-stress induced premature senescence by ANM;

FIG. 11 is a flowchart of a method of treating aging of cells by administering antcin M.

### DESCRIPTION OF THE PREFERRED EMBODIMENT

The following description of the preferred embodiment is provided to understand the features and the structures of the present invention.

In the present invention, a potent anti-aging compound is screened from a group of antcins and investigated the effects of ANM on SIPS in HNDFs by analyzing changes in the expression of the proteins. The effect of ANM is compared with known agents of N-acetylcysteine and resveratrol for anti-oxidant and SIRT-1 activation, respectively.

HG-Accelerated Growth Arrest and Senescence in HNDFs through Induction of ROS

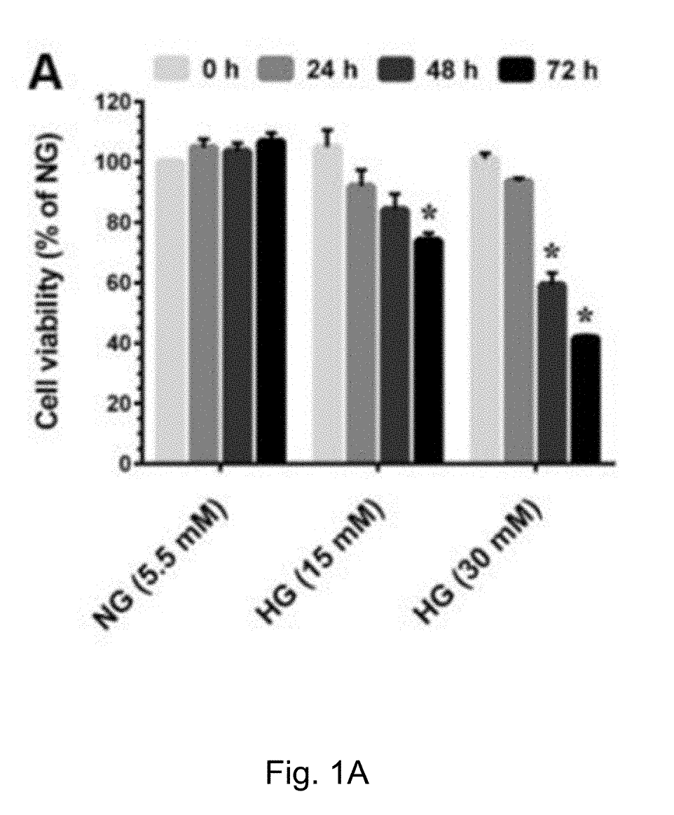

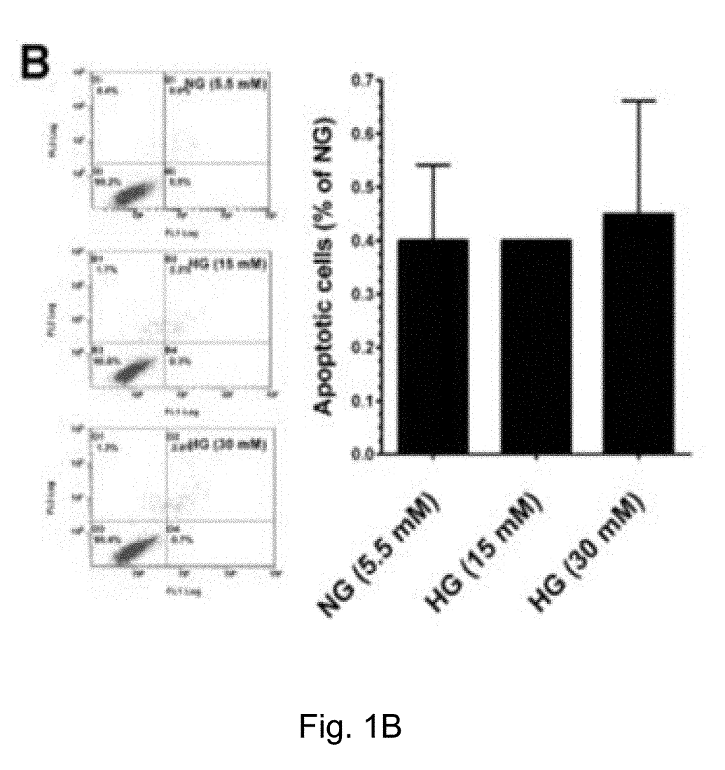

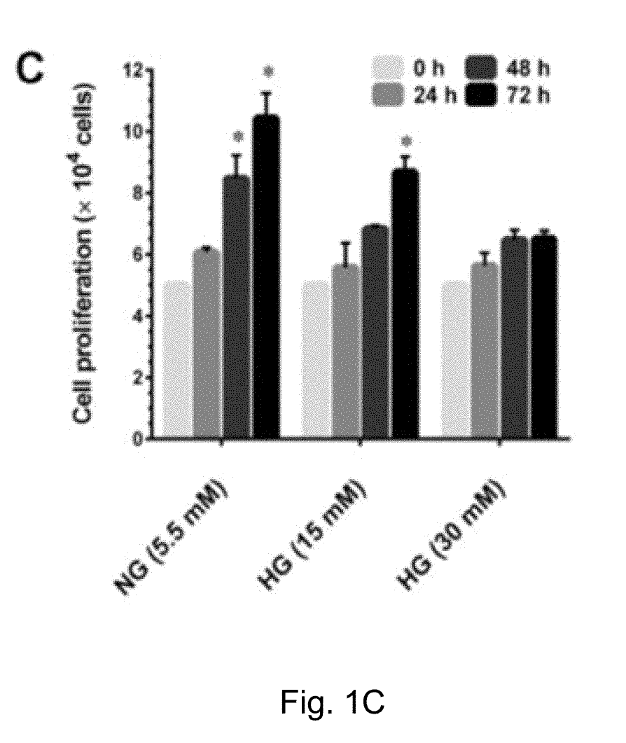

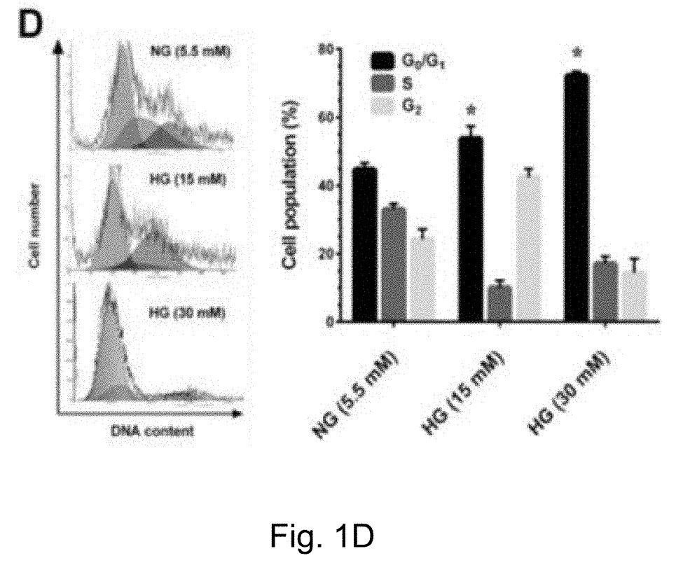

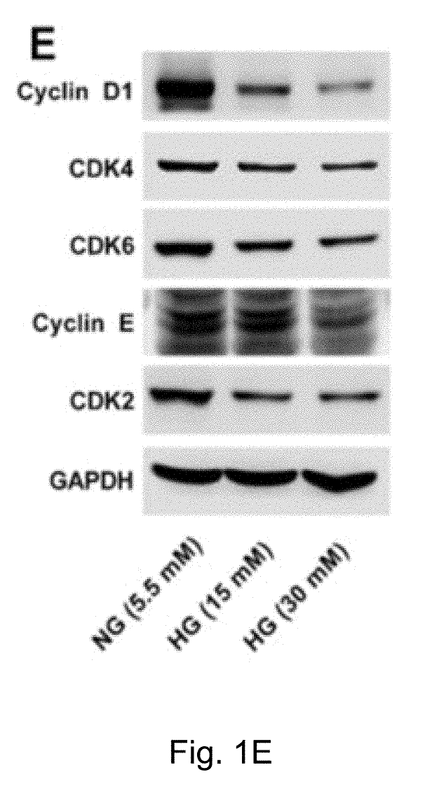

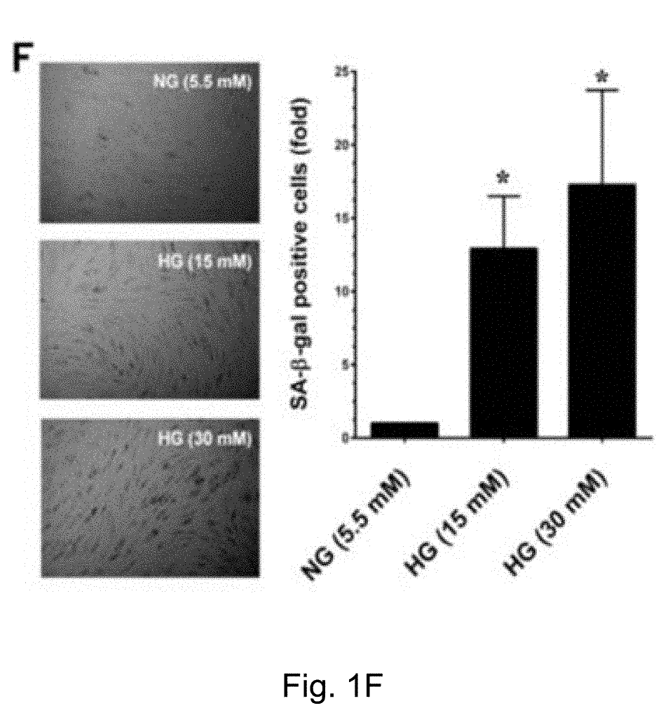

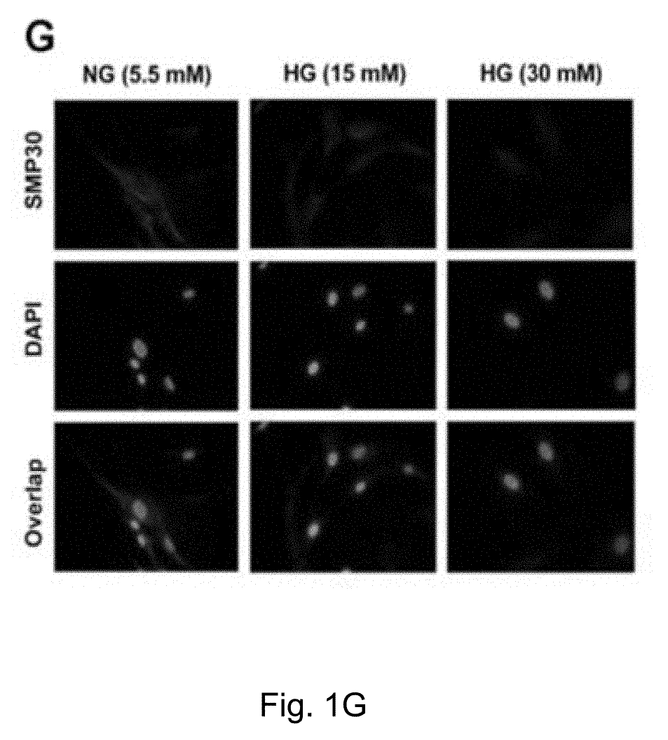

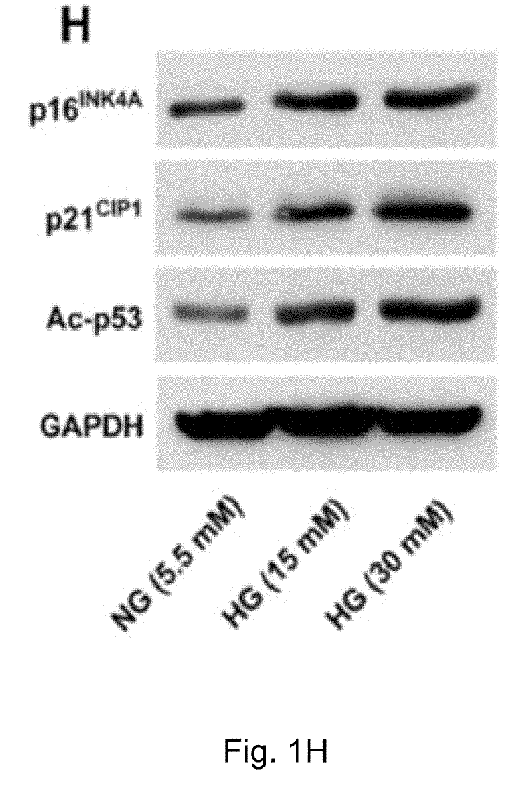

Please refer to FIG. 1A˜FIG. 1I, which are views showing stress-induced premature senescence accelerated by HG in dermal fibroblasts. As shown in the figures, to establish a human dermal fibroblast senescence model, an established oxidative stress-mediated senescence model is used, which involves incubating cells with HG (>30 millimoles (mM)) for 72 hours (h). To determine the cytotoxic effect of HG on the human dermal fibroblast-derived cell line CCD966SK, cells are incubated with increasing doses of HG (15 and 30 mM) for 24-72 h and the cell viability is measured by 3-(4,5-dimethyl-thiazol-2-yl)-2,5-diphenyl tetrazolium bromide (MTT) assay. Exposure to HG caused a dose- and time-dependent reduction in cell number. Particularly, treatment with high dose (30 mM HG) for 72 h, cell number is reduced to 41.7% (FIG. 1A).

Next, to examine whether the reduction in cell number is associated with apoptotic cell death, apoptosis is determined by Annexin-V/PI staining and the percents of cell death are shown in a histogram view. Results of flow cytometric analysis show that there is no significant increase in apoptotic-positive cells in HG treatment groups when compares to the control (NG) group (FIG. 1B). Therefore, it is supposed that HG induces growth arrest/senescence in fibroblasts, which is the reason for the reduction in cell number.

In FIG. 1C, to determine cell proliferation, 5×10⁴ cells/well of HNDFs are incubated in a 6-well plate with HG for 72 h. Number of viable cells are quantified by the tryphan blue exclusion method using a hemocytometer. Proliferation assay shows that, after treatment with HG (15 and 30 mM) for 72 h, there is sustained proliferation, which is equal to the initial seeding. Hence, HG causes dose- and time-dependent growth arrest in HNDFs.

To further clarify these results, cell-cycle analysis is performed. In FIG. 1D, cell-cycle distribution is measured by flow cytometer using propidium iodide (PI) and the percents of cells are shown in a histogram view. HNDFs treated with 15 and 30 mM of HG is arrested in the G1-S transition phase as evidenced by increased cell population in the G0/G1 phase from 46.1% (NG) to 51.1% and 73.1%, respectively.

Cell-cycle progression is further examined by quantifying cyclins and cyclin-dependent kinase (CDK) expression levels in HG-induced HNDFs. In FIG. 1E, immunoblotting is performed to determine the expression levels of cell-cycle regulatory proteins including cyclin D1, cyclin E, CDK4, CDK6 and CDK2, while GAPDH is served as an internal control. Results from immunoblotting strongly support the above observation that G1-S transition regulatory proteins such as cyclin D1, CDK4, CDK6, cyclin E and CDK2 are significantly down-regulated by HG in a dose-dependent manner compared with cells that had been cultured for the same time course in NG.

Accumulation of cells in the G0/G1 phase is one of the characteristic features of senescence. Therefore, a senescence-associated β-galactosidase (SA-β-gal) assay is used to examine whether HG induces senescence in HNDFs. In FIG. 1F, cellular senescence is determined by senescence-associated β-galactosidase (SA-β-gal) assay. The left panel shows representative figures and the right panel shows quantitative analysis of SA-β-gal positive cells per microscopic field. As is expected, an increased number of SA-β-gal positive cells are observed in HG-treated cells and this increase is noted in a dose-dependent manner.

Loss of senescence marker protein-30 (SMP30) expression is frequently observed in senescent cells. Hence, in FIG. 1G, immunofluroscence analysis shows expression and localization of SMP30 in HG-treated HNDFs. It is found that endogenous expression of SMP30 is significantly reduced by HG in a dose-dependent manner.

In addition, western blot analysis further supports an observation. In FIG. 1H, western blot analysis shows the protein expression levels of p16^{INK4A}, p21^{CIP1}, and p53 acetylation in HG-induced HNDFs. GAPDH served as an internal control. As in shown, senescence-associated modulation in proteins including p16^{INK4A}, p21^{CIP1} and acetylation of p53 are significantly increased by HG. HG increased intracellular reactive oxygen species (ROS), a major event triggering senescence, cell-cycle arrest and apoptosis in a variety of human cells.

To determine whether the HG-induced growth arrest and senescence are mediated by ROS, the intracellular ROS levels are measured by flow-cytometry using a DCF-DA probe. In FIG. 1I, intracellular ROS level is dertermined by flow cytometry using DCH-DA flurogenic probe. Left panel shows representative figures and the right pane shows quantitative analysis of intracellular ROS in HG-treated HNDFs. Results are expressed as mean±S.E.M of three independent expriments. Statistical significance is set at *P<0.05 compared to NG vs. HG. As is shown, treatment with 15 and 30 mM HG increases mean fluorescence intensity to nearly 3-fold of the oxidation-dependent fluorogen DCF-DA, which is proportional to the increase in intracellular ROS. Taken together, these data confirm that HG causes growth arrest and senescence in HNDFs without inducing cell death. Moreover, HG-induced ROS generation may play a crucial role in the onset of growth arrest and senescence in HNDFs. Furthermore, the present invention is the first in indicating hyperglycemia-induced oxidative stress-mediated senescence in HNDFs.

Screening of Anti-Aging Substances from Antrodia cinnamomea (A. cinnamomea) and Antrodia salmonea (A. salmonea)

Anticins are ergostane-type triterpenoids that have been reported to be anti-oxidant, anti-inflammatory and anti-cancer agents. In the present invention, an anti-aging agent is screened from a group of antcins including antcin A (ANA), antcin B (ANB), antcin C (ANC), antcinh (ANH), antcin K (ANK) and ANM.

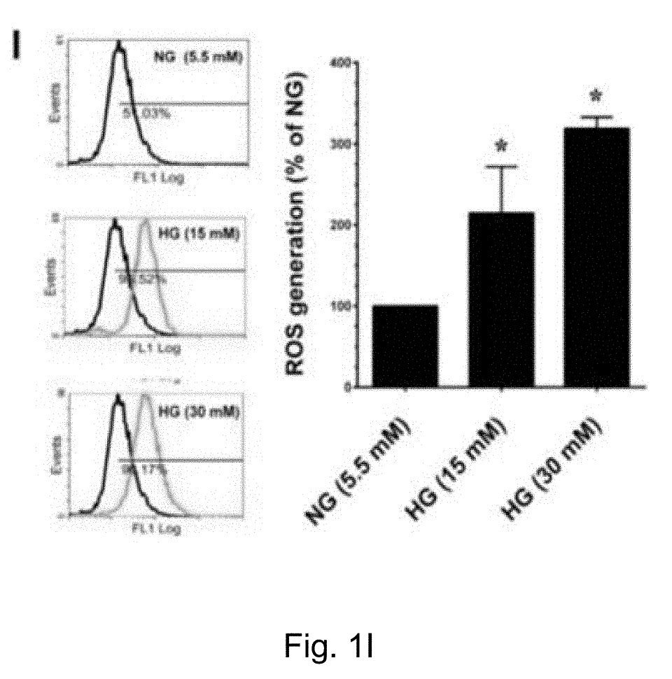

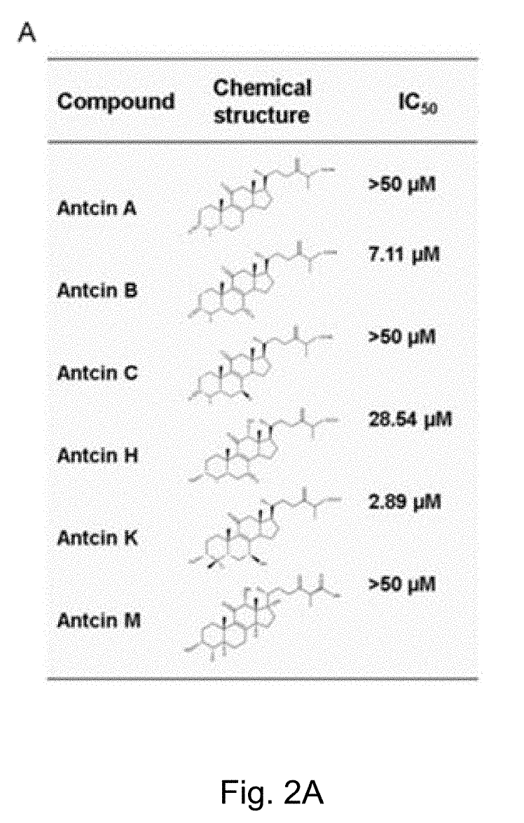

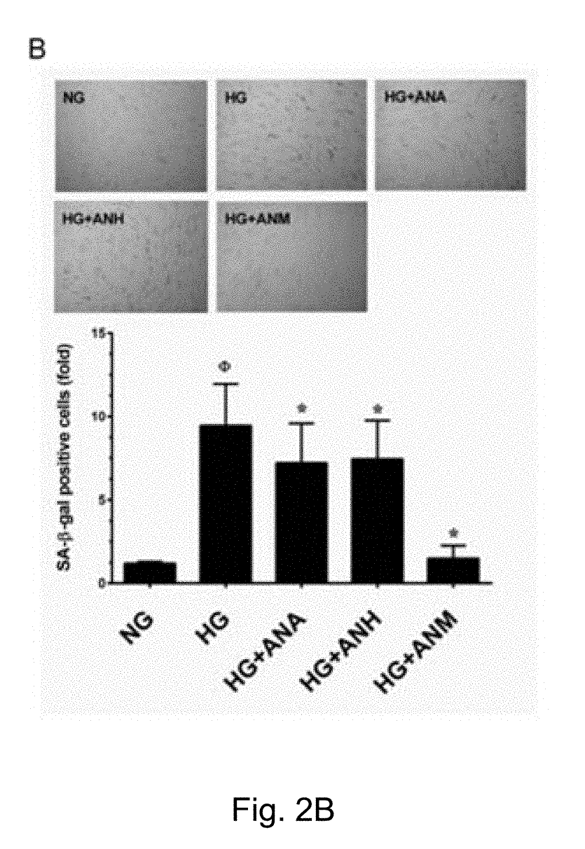

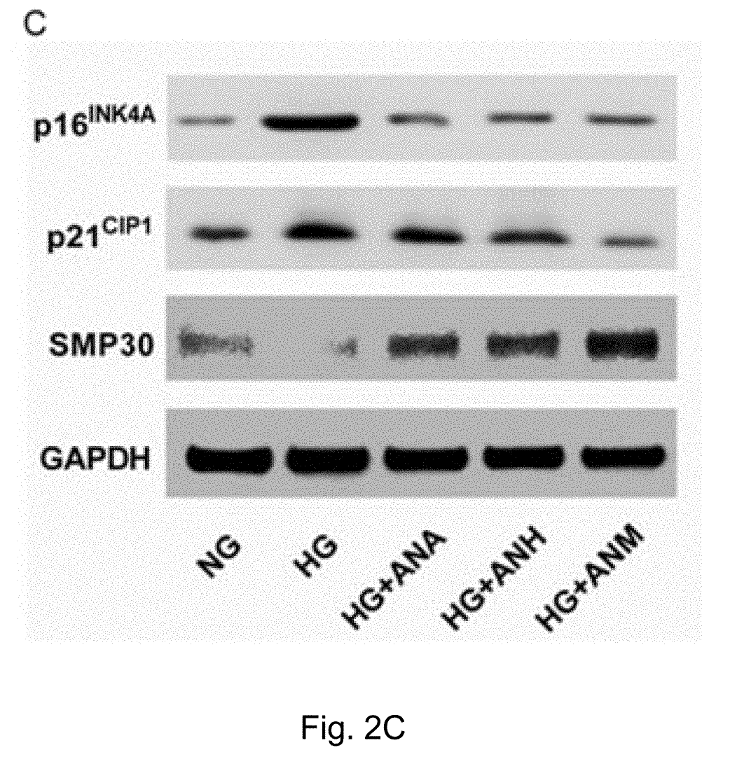

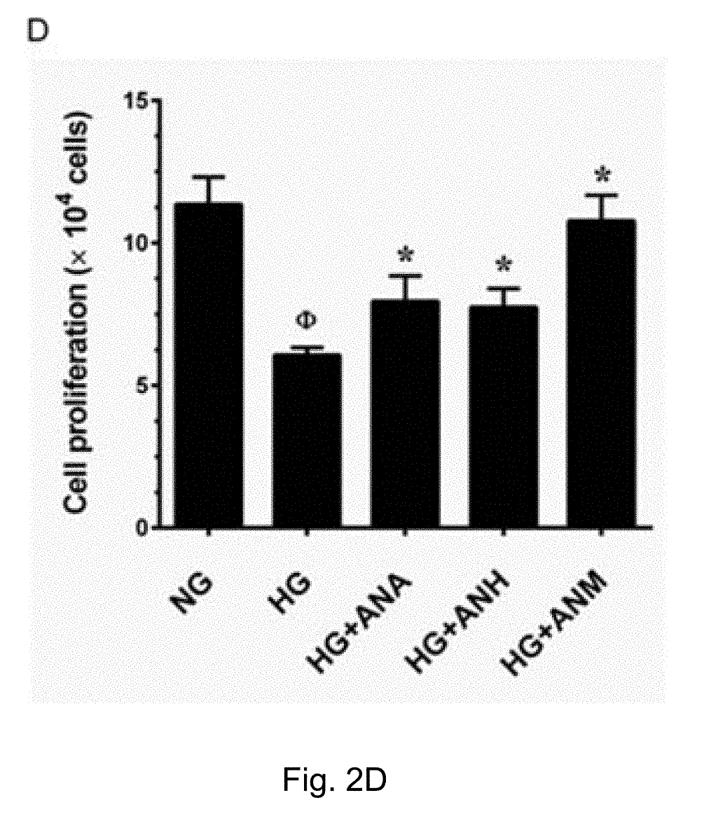

Please refer to FIG. 2A˜FIG. 2D, which are views showing HG-induced senescence affected by antcins in HNDFs. As shown in the figures, prior to an investigation, cytotoxicity of antcins against HNDFs is determined. In FIG. 2A, type of antcins are subjected to screen for potential anti-aging agents for skin aging. Names and chemical structures of the antcins are shown in the left and center column, respectively. The cytotoxic effects of antcins against HNDFs are investigated. Briefly, HNDFs are incubated with increasing concentrations (1, 5, 10 and 20 μM) of antcins including ANA, ANB, ANC, ANH, ANK and ANM for 72 h and the cell viability is determined by MTT assay. The 50% inhibitory concentrations (IC₅₀) of each compound against HNDFs are shown in right column. As is shown, ANB and ANK exhibited strong cytotoxicity to HNDFs with an IC₅₀ value of 7.11 and 2.89 μM, respectively. However, ANA, ANC, ANH and ANM did not show significant cytotoxicity to HNDFs up to the high treatment concentration (20 μM) and the IC₅₀ values are >50 μM.

Next, the protective effects of antcins on HG-induced HNDF senescence are examined. In FIG. 2B, cells were incubated with ANA (10 μM), ANH (10 μM) and ANM (10 μM) in the presence of HG (30 mM) for 72 h. Cellular senescence is determined by SA-β-gal activity. The left panel shows representative figures and the right panel shows quantitative analysis of SA-β-gal positive cells per microscopic field. After cells are co-incubated with HG and antcins (ANA, ANH and ANM) for 72 h, senescence is measured by SA-β-gal assay. Treatment with ANM shows significant protection against HG-induced HNDF senescence as evidenced by reduction in a number of SA-β-gal positive cells from 9.59-fold to 1.51-fold, whereas ANA and ANH show moderate inhibition as SA-β-gal positive cells are reduced to 7.46-fold and 8.13-fold, respectively.

In addition, in FIG. 2C, HNDFs are incubated with ANA (10 μM), ANH (10 μM) and ANM (10 μM) in the presence of HG (30 mM) for 72 h. Senescence-associated marker proteins such as p16^{INK4A}, p21^{CIP1} and SMP30 are determined by western blot analysis. Results from immunoblotting analysis confirm that HG-induced upregulation of senescence-associated proteins such as p16^{INK4A} and p21^{CIP1} are significantly downregulated by ANM, whereas ANA and ANH show a moderate inhibition, which is concomitant with the result of SA-β-gal assay. Moreover, compared with ANA or ANH, ANM rescues HG-induced SMP30 depletion in HNDFs where SMP30 is significantly upregulated.

To further clarify the effect of antcins, HG-induced reduction in cell proliferation is determined. In FIG. 2D, to determine the effect of antcins on cell proliferation efficacy, 5×10⁴ cells/well of HNDFs are incubated with antcins in 6-well plates in the presence of HG (30 mM) for 72 h. Number of viable cells is quantified by the tryphan blue exclusion method. Results are expressed as mean±S.E.M of three indipendent expriments. Statistical significance is set at ^{ϕ}P<0.05 as compared to NG vs. HG and *P<0.05 as compared to HG vs. samples. As is shown, a two-fold increase in cell proliferation is observed in the ANM treatment group, whereas ANA and ANH partially increase cell proliferation as compared to the HG treatment group. These data show that, out of the antcin group, ANM is a potent anti-aging component. Therefore, the molecular mechanism underlying the protective effect of ANM is explored next.

### HG-Induced Growth Arrest Blocked by ANM in HNDFs

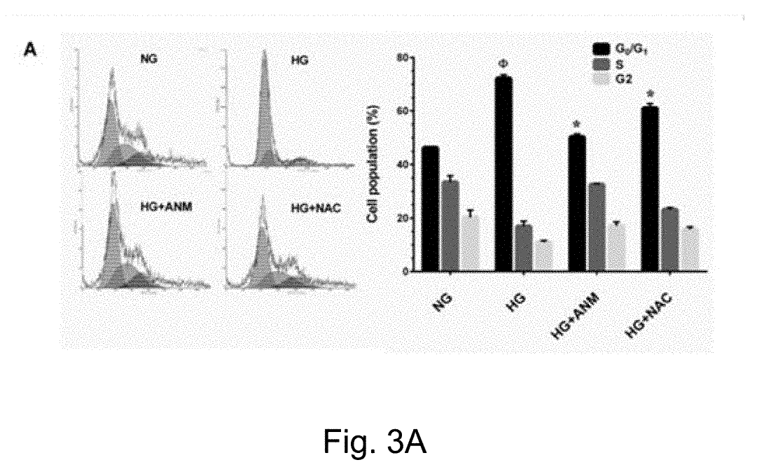

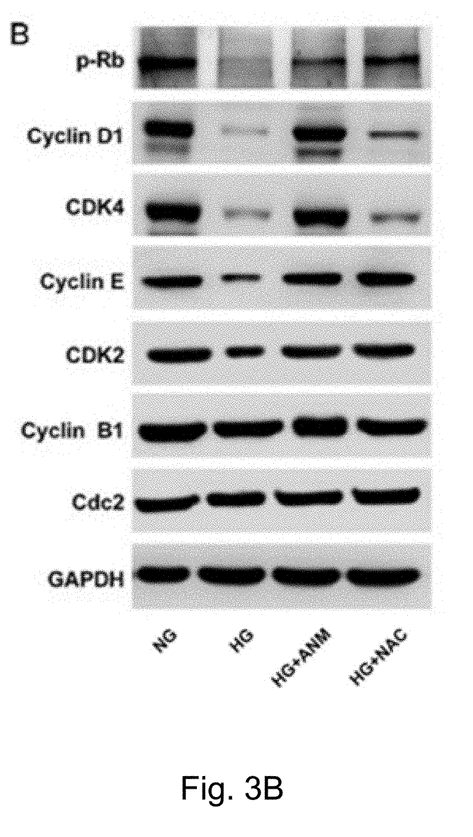

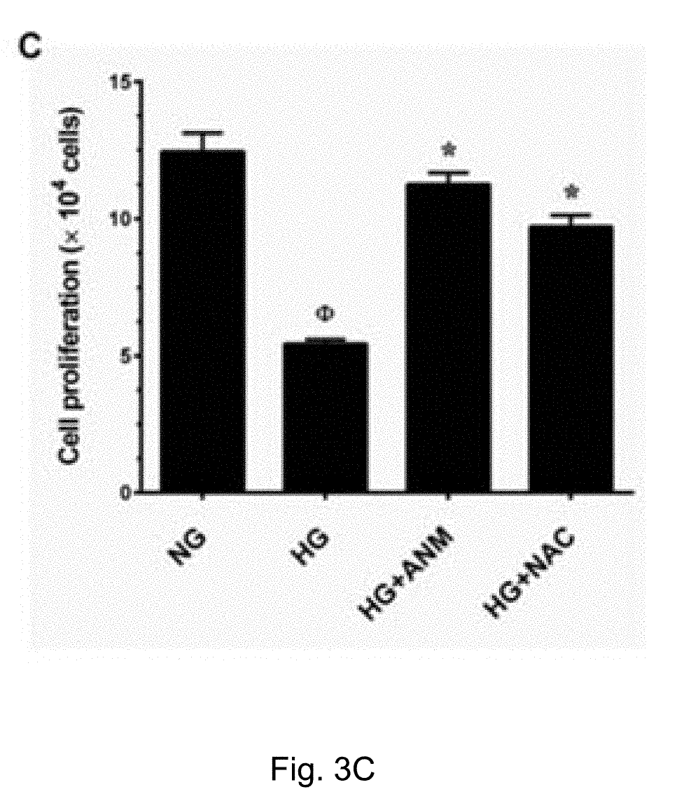

Senescence is well-defined as an irreversible arrest in the G0/G1 phase of the cell-cycle, triggered by various physiological and chemical stimuli including HG. It is thus paradoxical that HG-induced senescence is associated with cell-cycle arrest. Please refer to FIG. 3A˜FIG. 3C, which are views showing HG-induced growth arrest blocked by ANM in HNDFs. As shown in the figures, to further explore this paradoxical relationship, HNDFs are treated with HG and ANM or N-acetylcysteine (NAC). In FIG. 3A, HNDFs are incubated with ANM (10 μM) or NAC (100 μM) in the presence of HG (30 mM) for 72 h. Cell-cycle distribution is measured by flow cytometer using PI. Percentage of cell population in each transition phase is shown in a histogram view. The results demonstrate that treatment with HG causes cell-cycle arrest in the G1-S transition phase, as the proportion of cells in the G0/G1 phase is significantly increased to 71.2% as compared to 46.5% in the NG group. Treatment with ANM eliminates the effect of HG and reduces the cell population in the G0/G1 phase to 49.5%, which is similar to the control (NG) group. However, treatment with NAC partially blocks HG-induced cell-cycle arrest in HNDFs.

To further clarify this effect, G1-S transition regulatory proteins are determined by immunoblotting. In FIG. 3B, western blot analysis is performed to determine the expression levels of cell-cycle regulatory proteins including, pRb, cyclin D1, cyclin E, cyclin B1, CDK4, CDK6, CDK2 and Cdc2; and GAPDH is served as an internal control. As is shown, cells exposed to HG for 72 h result in a significant increase in protein Rb phosphorylation and a decrease in cyclin D1, CDK4, CDK6, cyclin E and CDK2 protein levels as compared to NG. However, treatment with ANM significantly inhibits protein Rb phosphorylation and upregulated cyclin D1, CDK4, CDK6, cyclin E and CDK2, whereas cyclin B1 and Cdc2 levels are unaffected. As mirroring the results of the flow cytometric analysis, immunoblotting also shows that treatment with NAC partially rescues HG-induced reduction in cyclins and CDKs. This result supports the observation above that treatment with ANM or NAC significantly rescues HG-mediated decrease in AKT or ERK1/2 phosphorylation, which plays a functional role in cell proliferation and survival.

Furthermore, in FIG. 3C, to determine the effect of ANM or NAC on cell proliferation, 5×10⁴ cells/well of HNDFs are incubated in a 6-well plate with ANM (10 μM) or NAC (100 μM) in the presence of HG (30 mM) for 72 h. Number of viable cells is quantified by the tryphan blue exclusion method. Results expressed as mean±S.E.M of three independent experiments. Statistical significance is set at ^{ϕ}P<0.05 as compared to NG vs. HG and *P<0.05 as compared to HG vs. samples. Cell proliferation analysis confirms that treatment with ANM protects HNDFs from HG-induced growth arrest, as indicated by increased cell proliferation.

HG-Induced Senescence Inhibited by ANM in HNDFs through Blocking ROS Generation

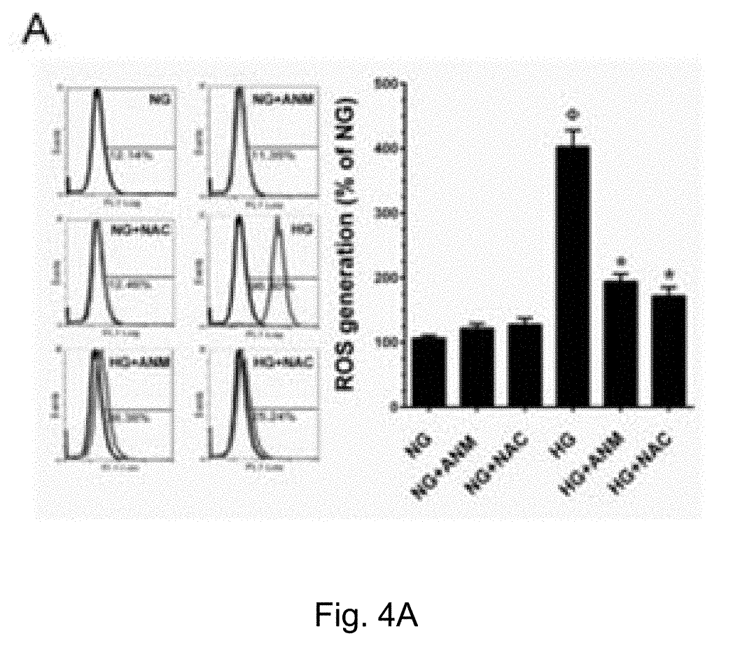

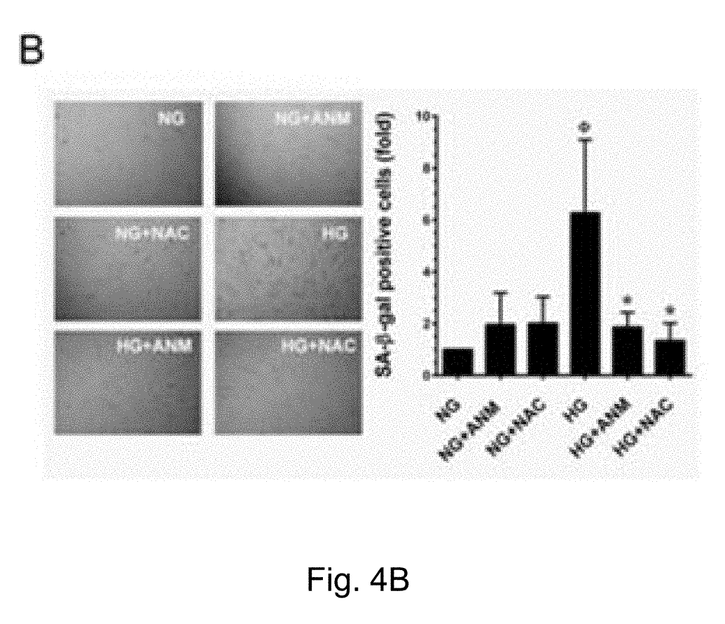

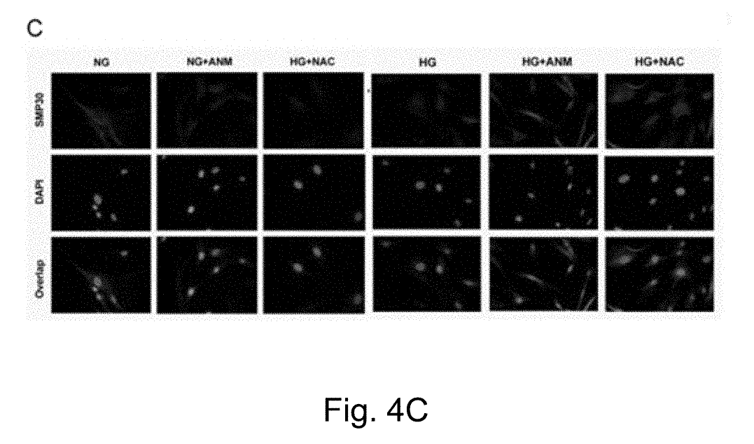

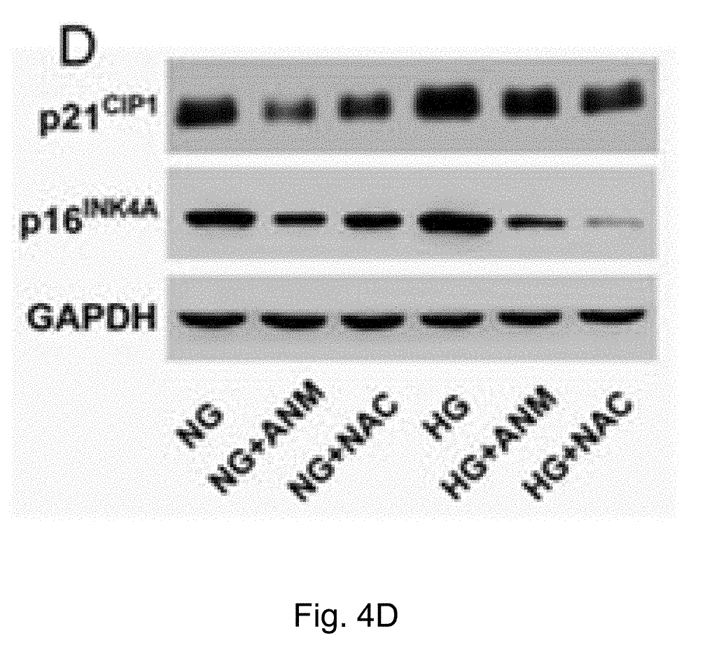

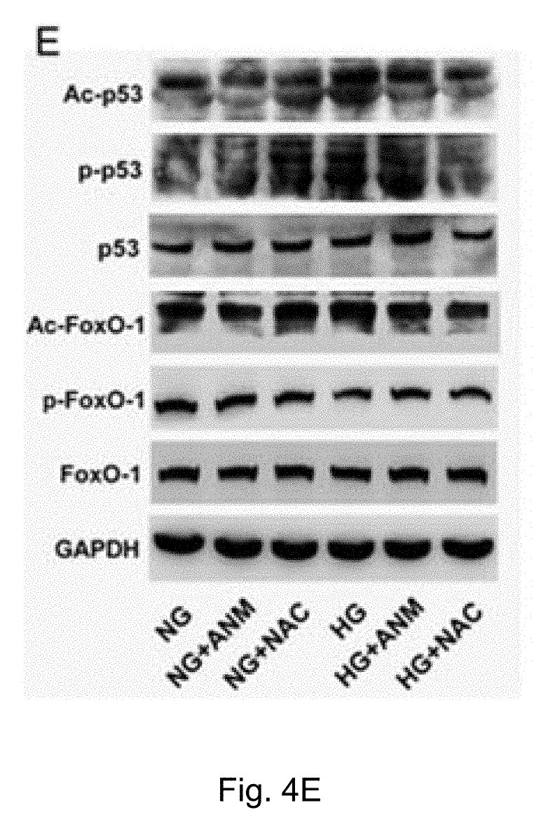

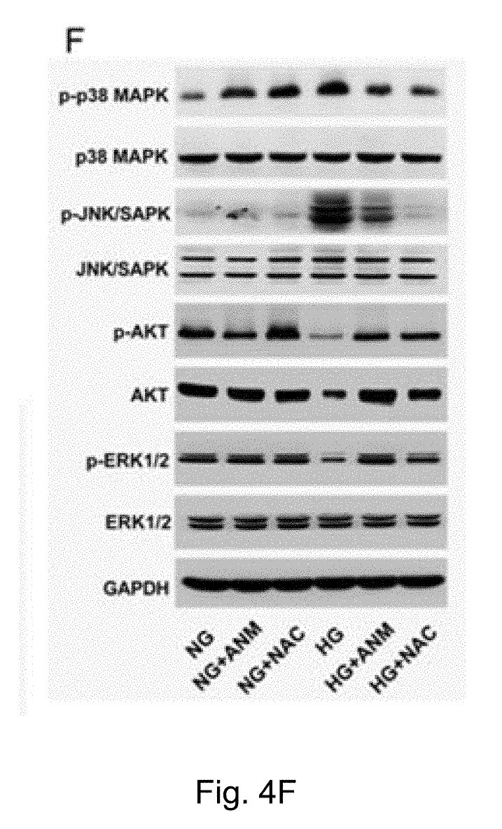

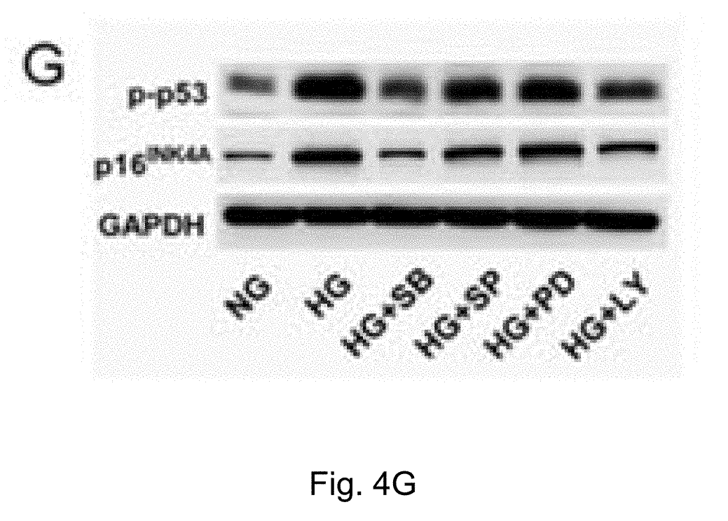

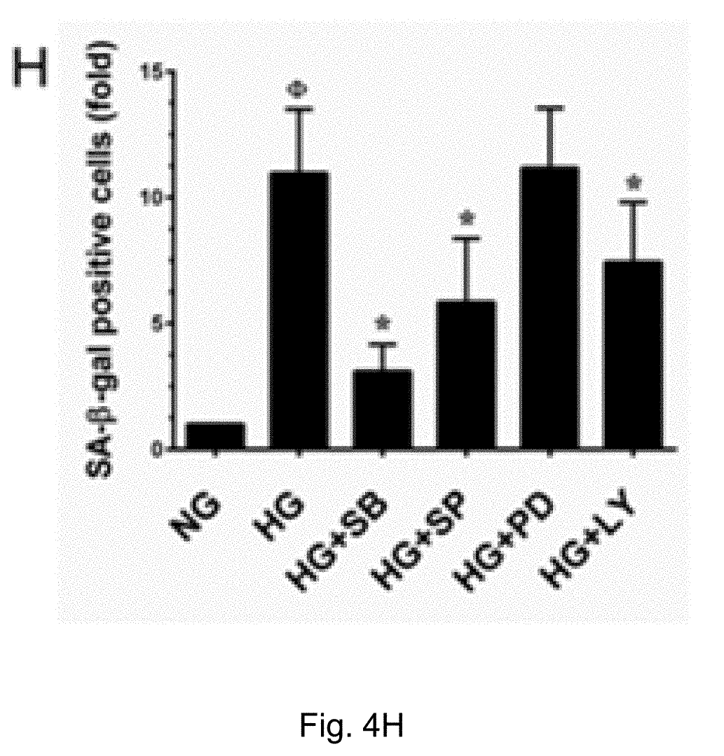

Next, please refer to FIG. 4A˜FIG. 4H, which are views showing HG-induced senescence inhibited by ANM in HNDFs. As shown in the figures, it is examined whether ANM inhibits HG-induced ROS generation. In FIG. 4A, HNDFs are incubated with ANM (10 μM) or NAC (100 μM) either in the presence or absence of HG (30 mM) for 24 h. The intracellular ROS level is determined by flow cytometry using DCH-DA flurogenic probe. The left panel shows representative figures and the right panel shows quantitative analysis of intracellular ROS in HG-treated HNDFs. As is shown, HNDFs are co-incubated with HG and ANM or NAC for 24 h, and intracellular ROS levels are measured by flow cytometry. Treatment with ANM or NAC alone does not significantly increase ROS generation, whereas HG-induced ROS generation (426.2%) is significantly prevented by ANM (200.8%) or NAC (176.58%).

Therefore, in order to examine whether the ROS inhibitory effect may be extended to suppress HG-induced senescence, HG-induced senescenece eliminated by ANM is examined. In FIG. 4B, HNDFs are incubated with ANM (10 μM) or NAC (100 μM) in the presence or absence of HG (30 mM) for 72 h. Cellular senescence is determined by SA-β-gal assay. The left panel shows representative figures and right panel shows quantitative analysis of SA-β-gal positive cells per microscopic field. As is shown, cells are co-incubated with HG and ANM or NAC for 72 h. Treatment with ANM significantly blocks the HG-induced senescence in HNDFs as evidenced by decreased number of SA-β-gal positive cells from 5.72-fold to 1.89-fold. A similar result is also observed in a pharmacological inhibitor of NAC.

Moreover, SMP30 has been considered to be an important protein marker of aging.

In FIG. 4C, SMP30 expression provided by ANM is examined. HNDFs are incubated with ANM (10 μM) or NAC (100 μM) in the presence or absence of HG (30 mM) for 72 h. The protein expression of SMP30 is measured by immunofluorescence using SMP30 specific primary antibody and fluorescein isothiocyanate-conjugated secondary antibody (green). The cellular localization of SMP30 is photographed using a fluorescence microscope. 4′,6-diamidino-2-phenylindole (DAPI) (1 μM) is used to stain the nucleus. Immunofluorescence analysis shows that HG-induced reduction in SMP30 is significantly prevented by ANM as compared with cells that are exposed to HG alone. In contrast, NAC partially prevented the HG-induced SMP30 depletion in HNDFs.

In order to assess the cellular and molecular basis of the ANM-mediated inhibition of senescence, the expression levels of senescence-associated marker proteins including, p16^{INK4A} and p21^{CIP1} are examined. In FIG. 4D, to determine the effect of ANM on senescence-associated protein expression, HNDFs are incubated with ANM (10 μM) or NAC (100 μM) in the presence or absence of HG (30 mM) for 72 h. Immunoblotting analysis is used to determine the protein levels with corresponding specific antibodies. Immunoblot analyses indicates that p16^{INK4A} and p21^{CIP1} protein levels are significantly increased in the HG treatment group as compared to the NG, while co-incubation with ANM significantly attenuates the expression levels of p16^{INK4A} and p21^{CIP1} proteins.

Indeed, treatment with ANM alone significantly reduces the basal level of p21^{CIP1} expression in HNDFs. It is well known that p16^{INK4A} and p21^{CIP1} are regulated by transcription factors p53 and FoxO1 followed by acetylation. In FIG. 4E, the result shows that treatment with HG markedly increases p53 and FoxO1 acetylation, whereas in the presence of ANM, acetylation in p53 and FoxO1 are barely observed. In addition, p53 phosphorylation at Ser15 by their upstream kinases promotes transcriptional activation in response to DNA damage. Here it is found that HG treatment results in a remarkable increase in p53 phosphorylation at Ser15, which is significantly blocked by ANM or NAC. Furthermore, the phosphorylation levels of FoxO1 (p-FoxO1) significantly decline in the HG treatment group, whereas co-treatment with ANM or NAC fails to protect against the decrease in FoxO1 phosphorylation.

In addition, neither ANM nor NAC affected the total p53 and FoxO1 levels. Next, the possible upstream regulators of p53 activation are examined. Previous studies have shown that p38 MAPK mediated p53 activation in response to intracellular ROS generation. In FIG. 4F, this effect is further extended to its upstream regulator p38 MAPK. Treatment with ANM or NAC significantly prevents the HG-induced activation of p38 MAPK in HNDFs. HG treatment also significantly increases JNK/SAPK phosphorylation; however, co-incubation with ANM or NAC significantly prevents JNK/SAPK activation in HNDFs. In addition, HG treatment caused a remarkable decrease in AKT and ERK1/2 activity, whereas ANM and NAC treatment significantly blocked this effect.

To further examine the phenomenon that HG-induced p53 activation is relayed by the p38 MAPK or JNK/SAPK cascade, cells are incubated with corresponding pharmacological inhibitors, SB203580, SP600125, PD98059 and LY294002 for p38MAPK, JNK/SAPK, ERK1/2 and P13K/AKT, respectively in the presence of HG. In FIG. 4G, the data show that p16^{INK4A} expression and p53 phosphorylation are reduced in p38 MAPK inhibitor-treated cells, and a partial reduction in p16^{INK4A} and p53 activity is found in JNK/SAPK and P13K/AKT inhibitor-treated cells, whereas treatment with ERK1/2 inhibitor fails to protect HG-induced p16^{INK4A} and p53 activity in HNDFs.

In FIG. 4H, p38 MAPK, JNK/SAPK and AKT triggers HG-induced senescence. HNDFs are exposed to HG (30 mM) in the presence or absence of p38 MAPK, JNK/SAPK, ERK1/2 and AKT inhibitors SB203580 (SB, 30 μM), SP600125 (SP, 30 μM), PD98059 (PD, 30 μM) and LY294002 (LY, 30 μM) for 72 h. Cellular senescence is determined by SA-β-gal activity assay. Results are expressed as mean±S.E.M of three independent experiments. Statistical significance is set at ^{ϕ}P<0.05 as compared to NG vs. HG and *P<0.05 as compared to HG vs. samples. This effect is further confirmed with SA-β-gal activity assay that shows that HG-induced SA-β-gal activity is barely observed in p38 MAPK and JNK/SAPK inhibitor-treated cells, whereas inhibition of ERK1/2 does not affect the HG-induced SA-β-gal activity. In contrast, inhibition of AKT also reduces the HG-induced SA-β-gal activity. These results suggest that the p38 MAPK, JNK/SAPK and P13K/AKT cascades play a functional role in HG-induced p16^{INK4A} and p53 activation and senescence, and also suggest that ANM-mediated inhibition of p16^{INK4A} and p53 activity may be associated with suppression of p38 MAPK and JNK/SAPK activation.

### Nrf2-Dependent Antioxidant Genes Activated by ANM in HNDFs

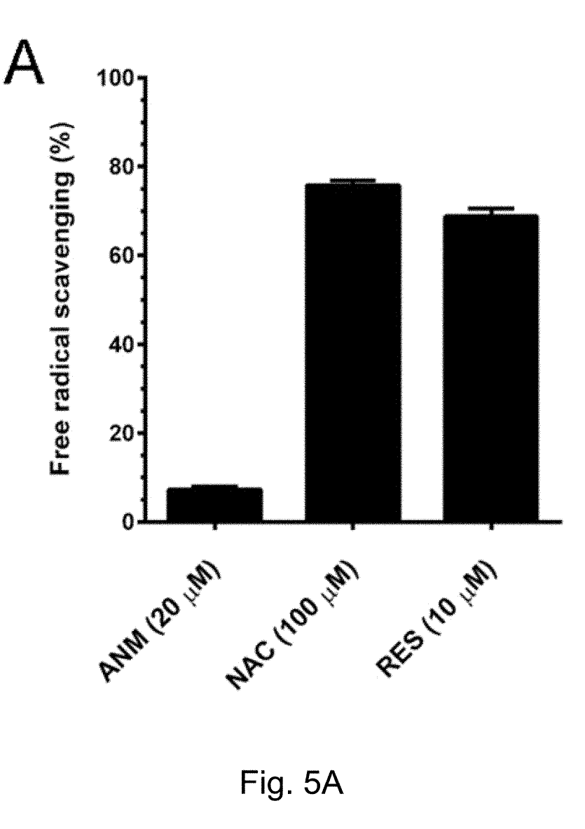

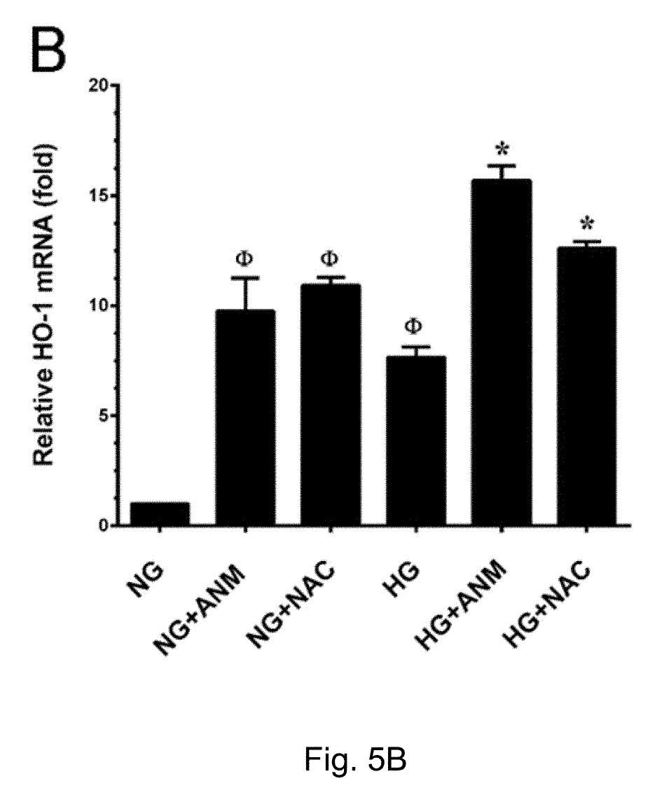

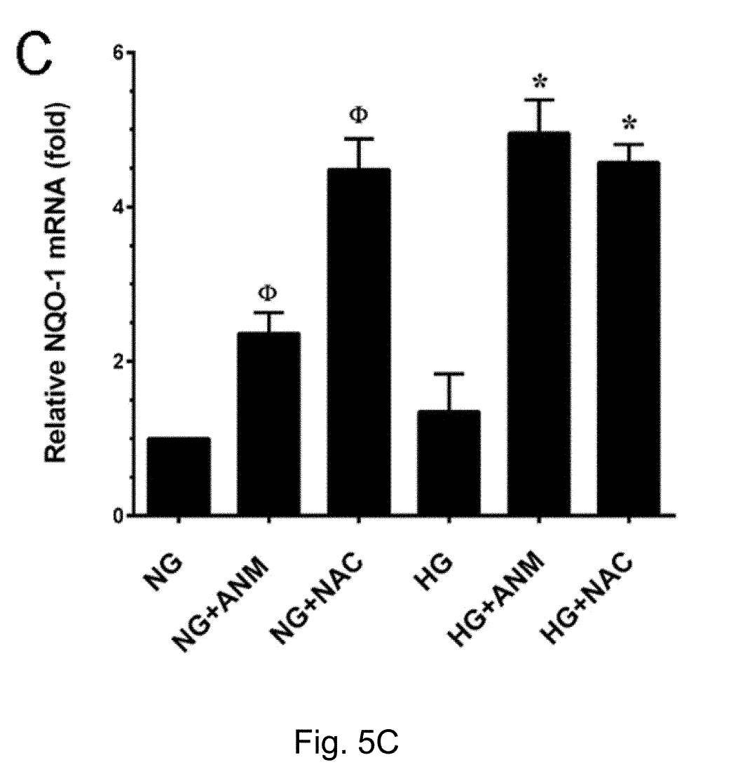

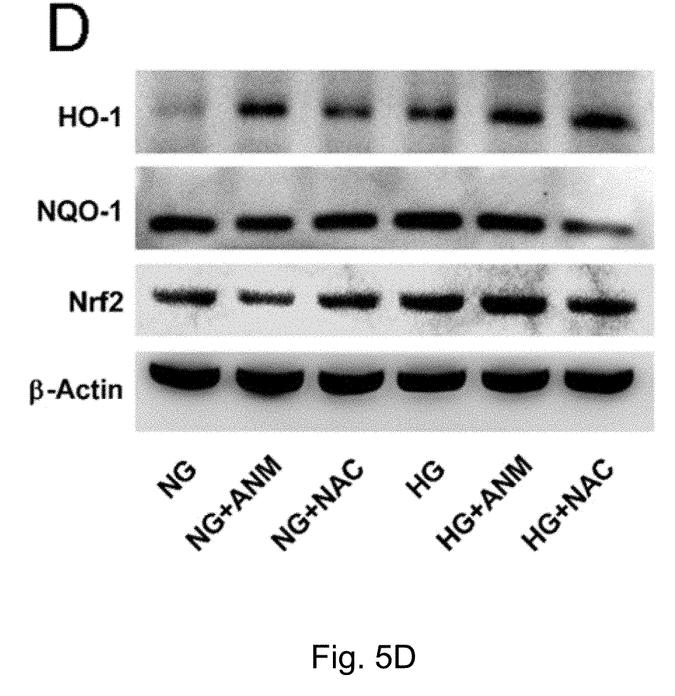

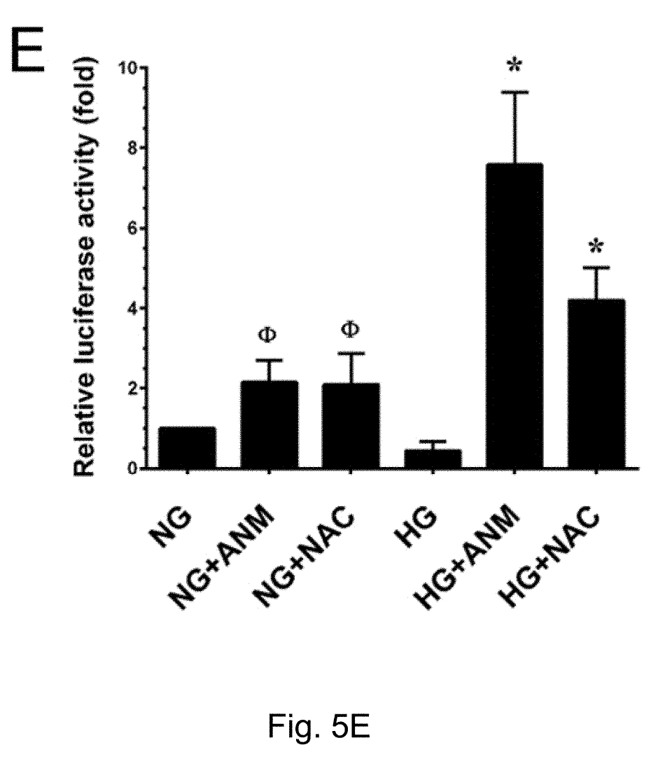

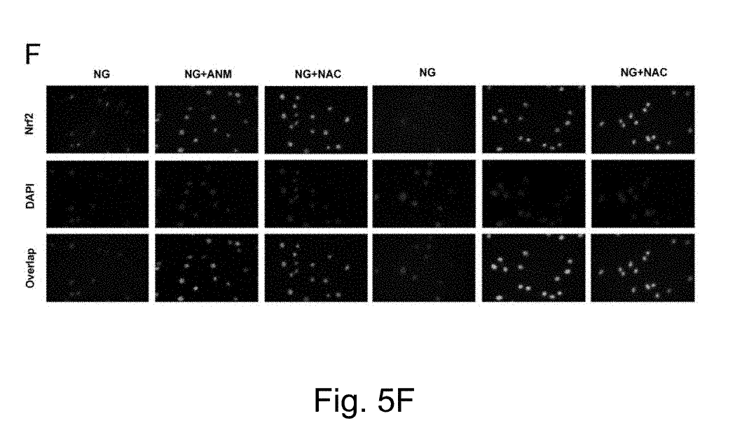

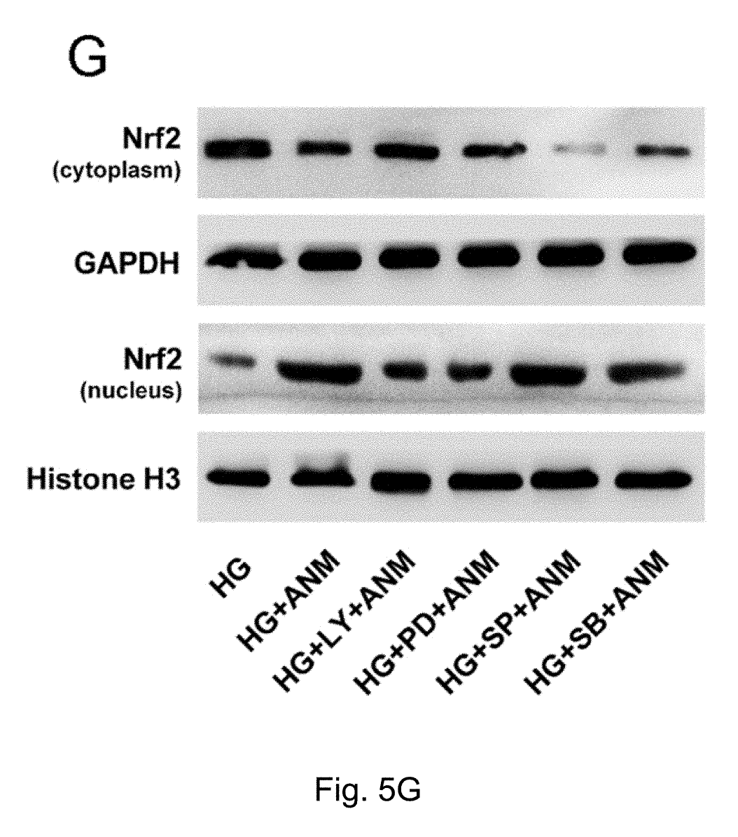

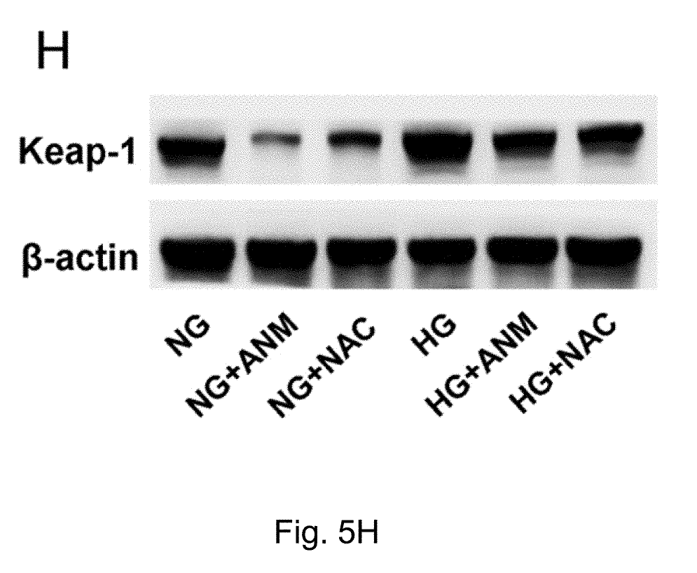

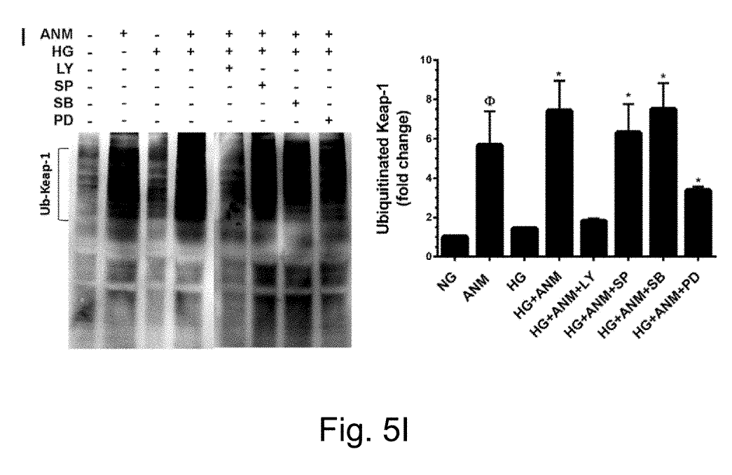

Please refer to FIG. 5A˜FIG. 5I, which are views showing Nrf2-dependent antioxidant genes activated by ANM in HNDFs. As shown in the figures, ANM inhibits ROS generation in HNDFs; however, the mechanism behind this activity is still unclear. Therefore, next to determine whether ANM acts directly as a free-radical scavenger, a cell-free DPPH free-radical scavenging assay is performed. In FIG. 5A, to determine the free-radical scavenging effect of ANM, cell-free DPPH assay is performed. NAC and RES are used as positive controls. As is shown, ANM fails to scavenge free radicals in the cell-free system, whereas NAC or resveratrol (RES) exhibit a potent free-radical scavenging effect.

In addition, it is reported that ANC, an analog of ANM, induces Nrf2-dependent anti-oxidant genes in hepatic cells

Therefore, it is hypothesized that ANM may upregulate anti-oxidant genes, which may suppress HG-induced ROS generation in HNDFs. In FIG. 5B and FIG. 5C, to quantify the mRNA expression levels of HO-1 and NQO-1, HNDFs are incubated with ANM (10 μM) or NAC (100 μM) in the presence or absence of HG (30 mM) for 12 h. Total RNA is extracted and subjected to Q-PCR analysis. Relative mRNA levels are normalized with β-actin mRNA. As is expected, treatment with ANM significantly increases the mRNA levels of phase II enzymes such as HO-1 and NQO-1 in HNDFs. In contrast, as compared with the NG treatment group, increased expression levels of HO-1 and NQO-1 are observed in the HG treatment group. However, treatment with ANM further increased HO-1 and NQO-1 in the HG treated groups.

In FIG. 5D, to determine the protein expression levels of HO-1, NQO-1 and Nrf2, HNDFs are incubated with ANM (10 μM) or NAC (100 μM) for 24 h. Total cell lysates are prepared and subjected to western blot analysis to monitor the expression levels of HO-1, NQO-1 and Nrf2. The result is further confirmed by western blotting which demonstrates that, as compared to the control (NG), ANM and NAC significantly increase HO-1 expression in both the NG and HG groups, whereas NQO-1 is unaffected by both ANM and NAC.

It is well demonstrated that anti-oxidant genes including HO-1 and NQO-1 are regulated by the transcription factor Nrf2. Therefore, to determine whether ANM augments Nrf2 transcriptional activity, ARE-harboring luciferase reporter assay is used. In FIG. 5E, to determine the Nrf2 transcriptional activity, HNDFs are transiently transfected with ARE promoter construct using lipofectamine and incubated with ANM (10 μM) or NAC (100 μM) in the presence or absence of HG (30 mM) for 6 h. Cell lysates are mixed with luciferase reagents and quantified using an illuminometer. Relative ARE promoter activity is calculated by dividing the relative luciferace unit (RLU) of treated cells by RLU of untreated cells (NG). As is shown, the luciferase activity in HNDFs transfected with the ARE reporter construct is significantly increased to 5.8-fold, 6.3-fold and 2.5-fold by ANM, NAC and HG, respectively, as compared to the control (1-fold). However, a remarkable increase in luciferase activity is observed in cells that are co-treated with HG and ANM or NAC which showed a 8.5-fold and 8.2-fold increase, respectively.

Transcriptional activation of Nrf2 is dependent upon the rate of nuclear export followed by disassociation from cytoplasmic Keap-1. In FIG. 5F, to determine the nuclear localization of Nrf2, HNDFs are incubated with ANM (10 μM) or NAC (100 μM) in the presence or absence or HG (30 mM) for 2 h. The protein expression and localization of Nrf2 are measured by immunofluorescence using Nrf2 specific primary antibody and fluorescein isothiocyanate-conjugated secondary antibody (green). The subcellular and nuclear localization of Nrf2 is photographed using a fluoroscence microscope. DAPI (1 μM) is used to stain the nucleus. Results from immunofluorescence analyses show that Nrf2 expression in the nucleus is barely observed in the control (NG) and the HG treatment groups, whereas elevated Nrf2 expression in the nucleus is observed in the ANM or NAC treatment groups.

Activation of P13K/AKT and mitogen-activated protein kinases (MAPKs), including ERK1/2, JNK/SAPK and p38 MAPK, facilitates Nrf2 transcriptional activation in a variety of human cell lines. In the above, it is indicated that ANM significantly increases AKT and ER1/2 activities, and decreases p38 MAPK and JNK/SAPK activities (as shown in FIG. 4F). To elucidate the upstream signaling events involved in ANM-induced Nrf2 transcriptional activity, cells are pre-incubated with pharmacological inhibitors of P13K/AKT (LY294002), ERK1/2 (PD98059), SAPK/JNK (SP600125) and p38MAPK (SB203580) for 2 h and treated with ANM for 6 h in the presence of HG. In FIG. 5G, HNDFs are pre-incubated with AKT, ERK1/2, JNK/SAPK and p38 MAPK inhibitors, including LY294002 (LY, 30 μM), PD98059 (PD, 30 μM), SP600125 (SP, 30 μM) and SB203580 (SB, 30 μM), respectively, for 2 h and then incubated with ANM (10 μM) in the presence of HG (30 mM) for 6 h. Cytoplasmic and nuclear fractions are prepared and subjected to western blot analysis. GAPDH and histone H3 are served as internal controls for the cytoplasmic and nuclear fraction, respectively. As is shown, in the ARE-dependent luciferase reporter system, pretreatment of cells with LY294002 and PD98059 effectively suppresses ANM-induced ARE luciferase activity, whereas pre-incubation of cells with SP600125 and SB203580 partially or barely inhibits luciferase activity. These results suggest that ANM-induced Nrf2 transcriptional activity is regulated by the activation of AKT or ERK1/2 in HNDFs.

Under normal physiological condition, Nrf2 is sequestered in the cytoplasm, where it associated with Keap-1, an actin-binding protein. Upon chemical treatment or oxidative stress conditions, the steady-state levels of Keap-1 is rapidly degraded through the ubiquitin-dependent proteasome pathway, which eventually causes Nrf2 accumulation and transcriptional activity. To determine whether the up-regulated ratio of Nrf2 in nucleus by ANM is due to the induction of Keap-1 ubiquitination, we examined the ubiquitination of Keap-1 by immunoprecipitation after treatment with ANM in the presence or absence of HG. In FIG. 5H, the Keap-1 protein expression level is determined by western blotting. The Keap-1 protein level is significantly decreased after treatment with ANM alone or in HG-induced condition. In the other hand, a significant increase in ubiquitination of total protein is observed in cells treatment with ANM or NAC.

In FIG. 5I, effect of ANM on ubiquitination of Keap-1 is examined. Equivalent amount of proteins are immune-precipitated with Keap-1 antibody and visualized by western blotting with ubiquitin antibody. Histogram shows the percentage of ubiquinated Keap-1. Results are expressed as mean±S.E.M of three independent experiments. Statistical significance is set at ^{ϕ}P<0.05 as compared to NG vs. HG or ANM alone or NAC alone and *P<0.05 as compared to HG vs. samples. As is shown, after immunoprecipitation with anti-Keap-1 antibody, a remarkable increase of ubiquitination of Keap-1 is observed in cells treatment with ANM, and further increase is observed when cells are co-incubated with HG. This data suggest that up-regulation of Nrf2 protein by ANM is due to the enhancement of Keap-1 ubiquitination, and has the possibility that ANM may directly or indirectly induce Keap-1 ubiquitination. The data also indicate that up-regulation of Nrf2 is mediated by AKT and ERK1/2 (as shown in FIG. 5G). Therefore, it is further examined whether AKT and ERK1/2 have any influence on Keap-1 ubiquitination. Cells are co-incubated with AKT, p38MAPK, JNK and ERK1/2 inhibitors in the presence of ATM; and the Keap-1 ubiquitination is examined. As shown in FIG. 5I, ANM-induced Keap-1 ubiquitination is markedly observed in p38MAPK or JNK1/2 inhibitors treated cells, whereas a reduced levels of Keap-1 ubiquitination is noted in AKT and ERK inhibitor treated cells. These data confirm that ANM-induced activation of AKT or ERK1/2 induces Keap-1 proteasome degradation in HNDFs.

### HG-Induced Oxidative Stress in Nrf2 Silenced Cells Failed to be Protected by ANM

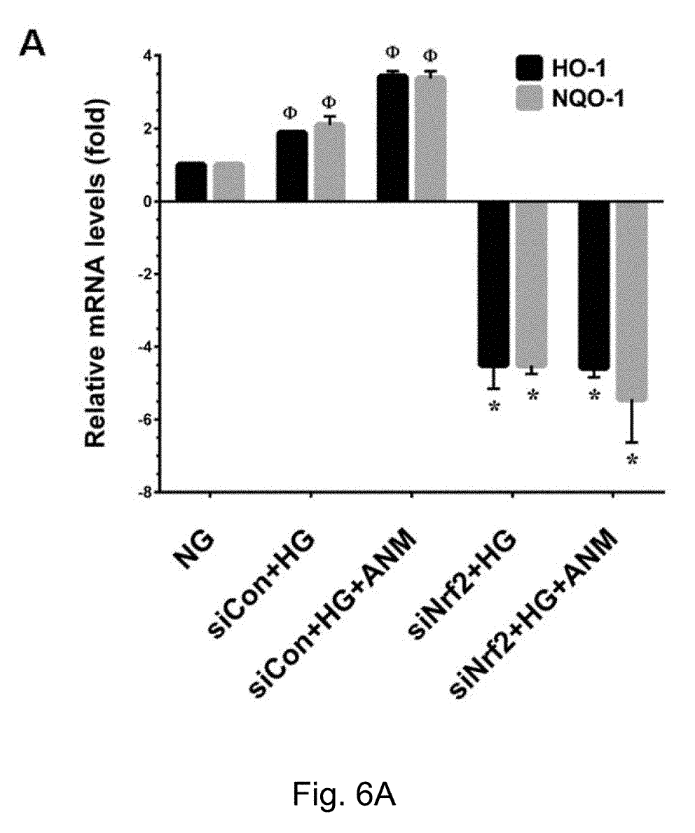

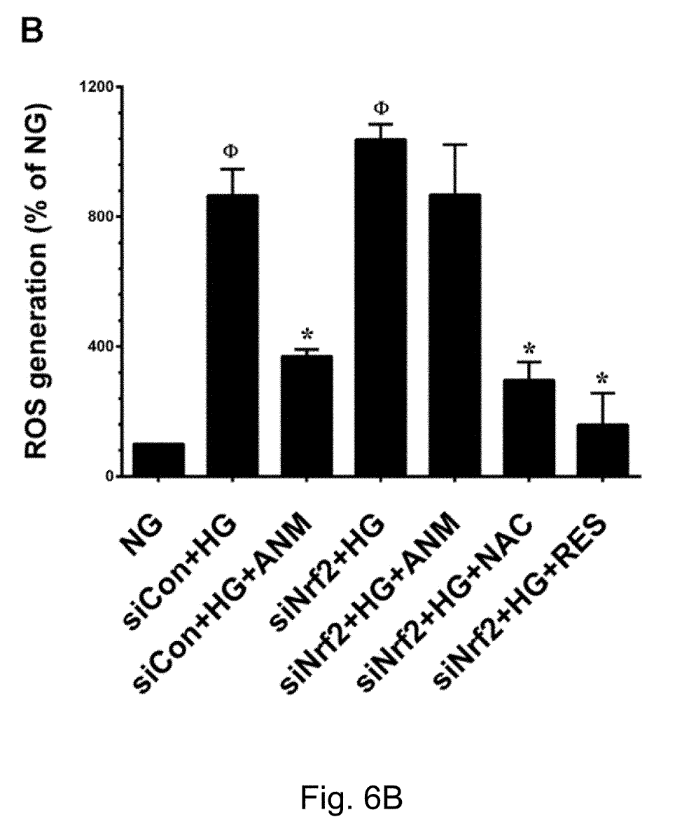

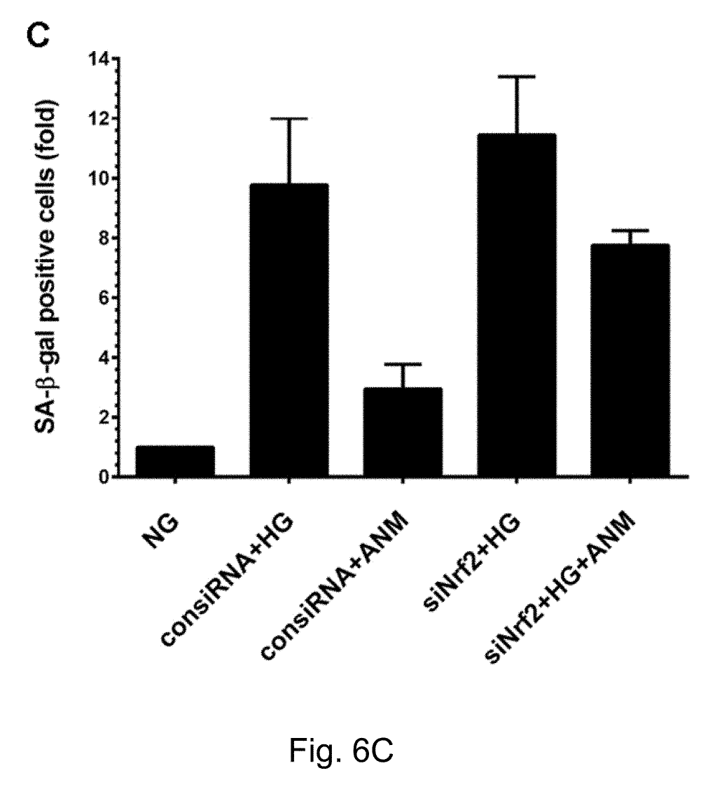

Please refer to FIG. 6A˜FIG. 6C, which are views showing HG-induced oxidative stress in Nrf2 silenced cells failed to be protected by ANM. As shown in the figures, to confirm a hypothesis that ANM protects HNDFs from HG-induced oxidative stress, an Nrf2 gene knockdown system using Nrf2 siRNA is developed. In FIG. 6A, HNDFs are transfected with specific siRNA against Nrf2 or control siRNA. After transfection for 24 h, cells are incubated with ANM (10 μM) in the presence of HG for 12 h. Total RNA is extracted and subjected to Q-PCR analysis to determine HO-1 and NQO-1 mRNA expression levels. As is shown, a partial increase in the expression levels of HO-1 and NQO-1 mRNA are observed in scrambled siRNA (control siRNA) transfected cells, and co-incubation with ANM exhibits a remarkable increase in HO-1 and NQO-1 mRNA levels. Although treatment with HG alone or along with ANM shows a decrease in HO-1 and NQO-1 expression in siNrf2-transfected cells, indeed, the HO-1 and NQO-1 mRNA levels decline below the basal level in siNrf2 transfected cells. From the data, it can be concluded that Nrf2 plays a vital role in HO-1 and NQO-1 induction even at the basal level.

Moreover, in FIG. 6B, HNDFs are transfected with specific siRNA against Nrf2 or control siRNA. After transfection for 24 h, cells are incubated with ANM (10 μM) or NAC (100 μM) or RES (5 μM) in the presence of HG for 24 h. Intracellular ROS is measured by DCFH-DA assay. As is shown, treatment with ANM significantly inhibits HG-induced ROS generation in scrambled siRNA transfected cells, whereas increased ROS generation is observed in siNrf2 transfected cells even after treatment with ANM.

To further clarify the protective effect, HG-induced senescence is measured by SA-β-gal assay. In FIG. 6C, HNDFs are transfected with specific siRNA against Nrf2 or control siRNA. After transfection for 24 h, cells are incubated with ANM (10 μM) in the presence of HG for 72 h. SA-β-gal activity is measured. Results are expressed as mean±S.E.M of three independent experiments. Statistical significance is set at ^{ϕ}P<0.05 as compared to NG vs. HG or ANM alone or NAC alone and *P<0.05 as compared to HG vs. samples. As is shown, in control siRNA transfected cells, treatment with ANM significantly inhibits HG-induced senescence. In contrast, treatment with ANM significantly prevents HG-induced senescence in siNrf2-transfected cells. The data suggest that ANM-induced activation of the Nrf2-dependent antioxidant mechanism at least partially supports the protective effect of ANM; however, there may be other possible mechanisms involved in the complete protection provided by ANM.

### SIRT-1 Upregulated by ANM in HNDFs

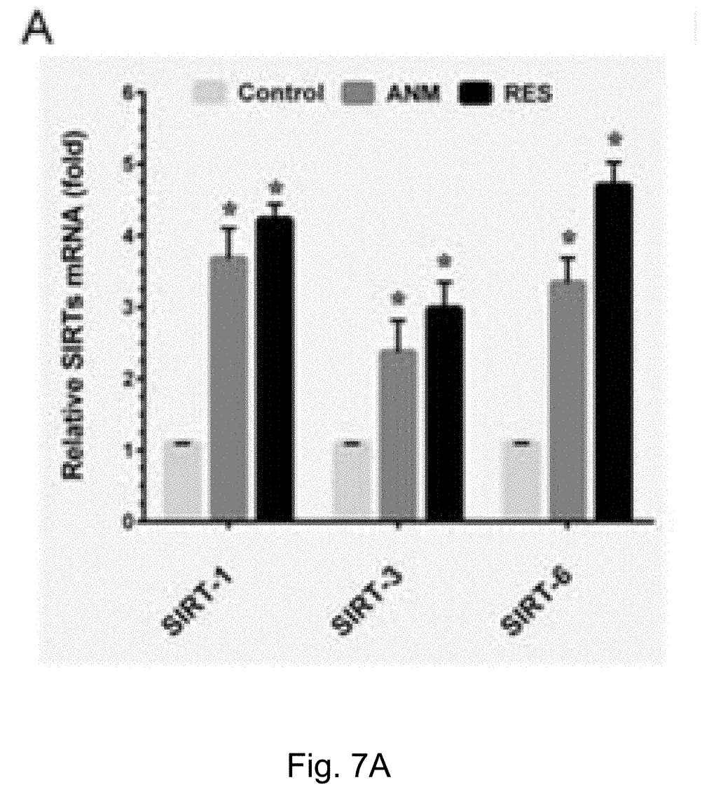

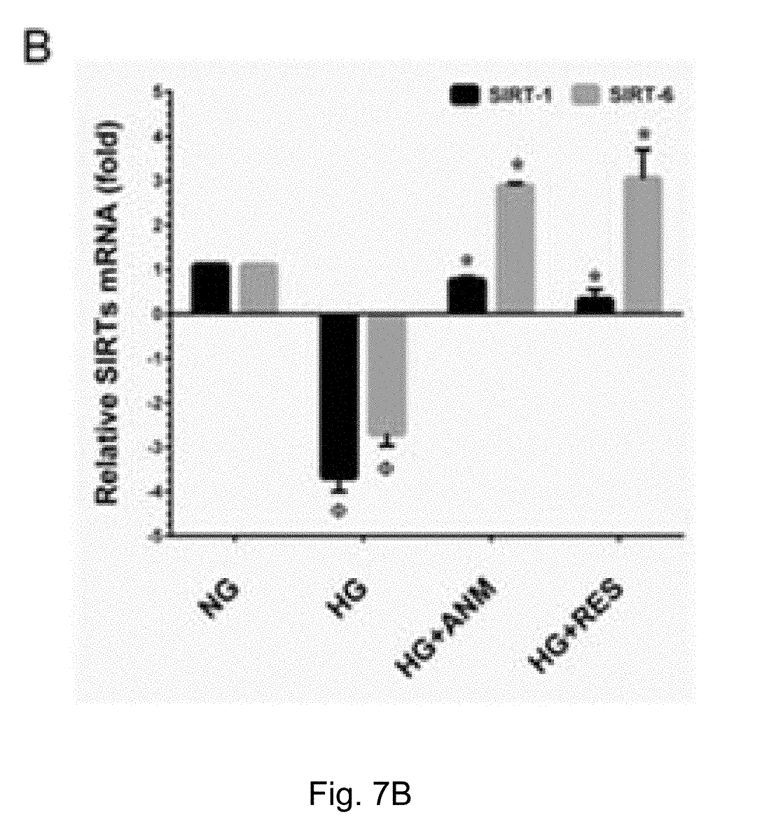

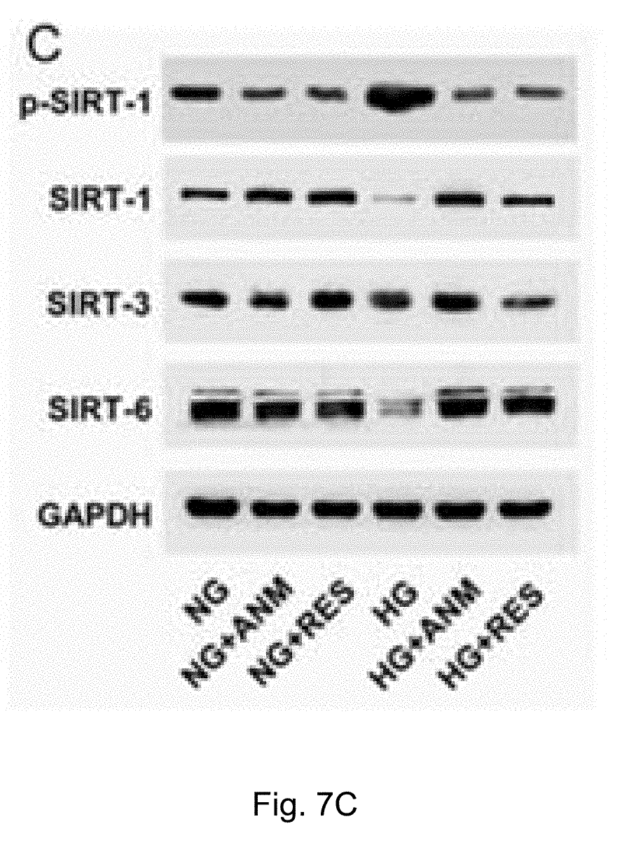

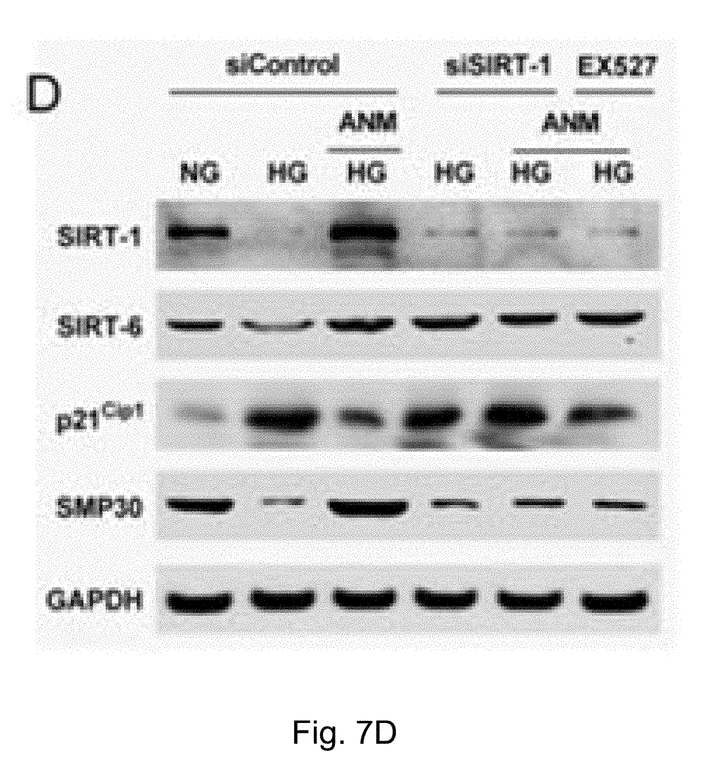

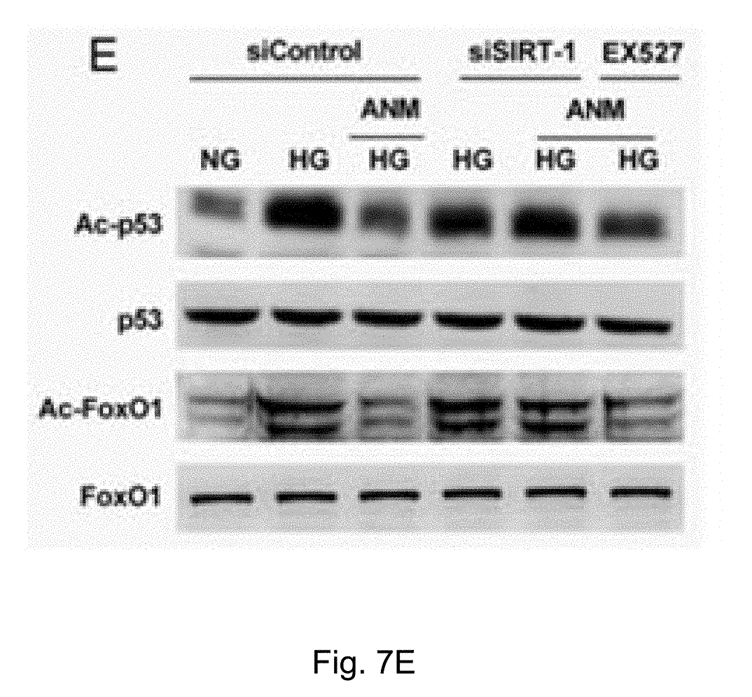

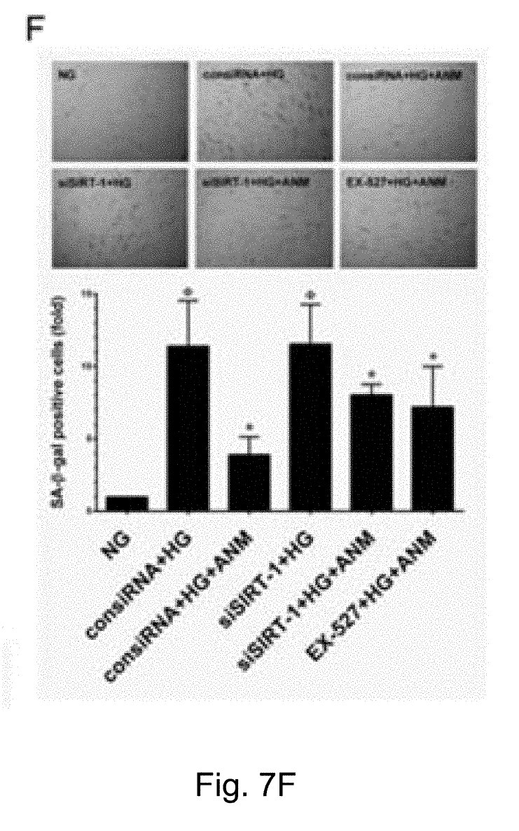

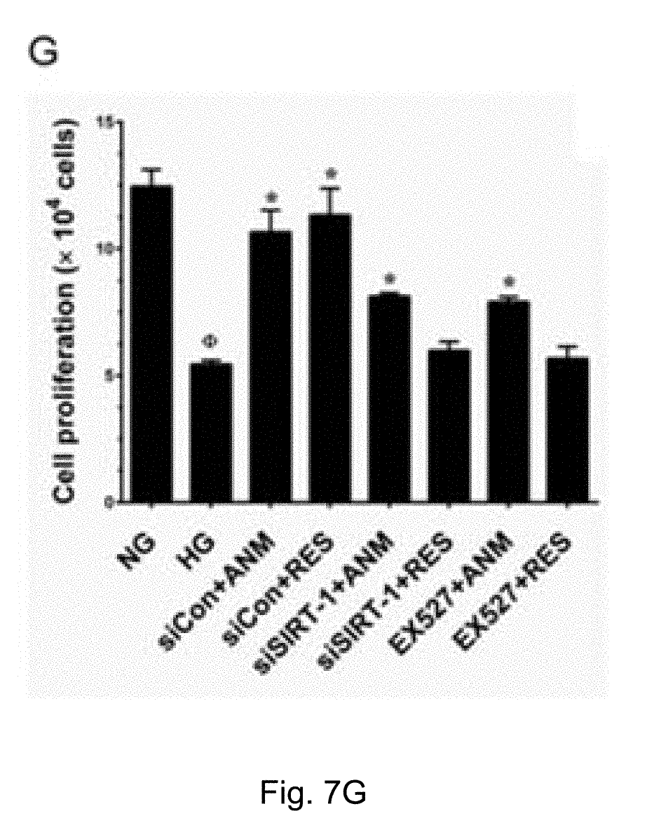

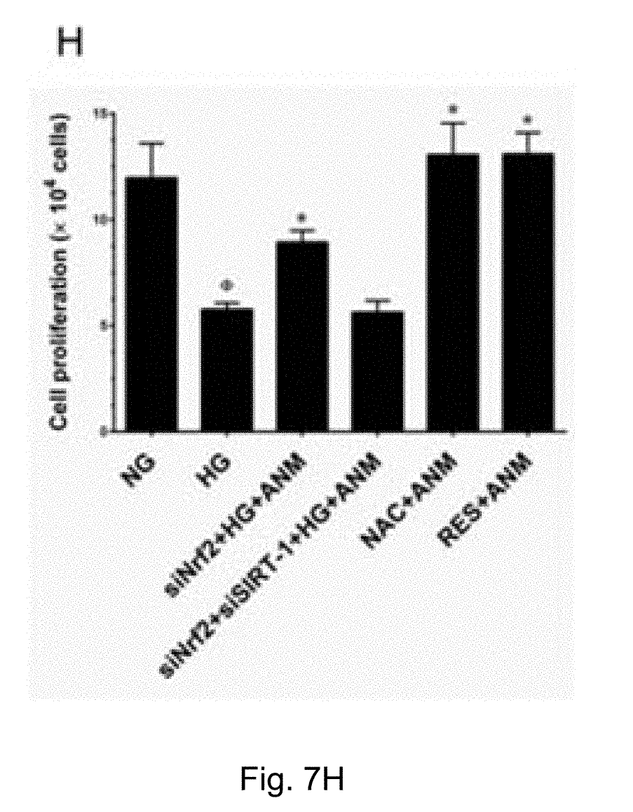

Please refer to FIG. 7A˜FIG. 7I, which are views showing silent mating type information regulation 2 homologs 1 (SIRT-1) upregulated by ANM in HNDFs. As shown in the figures, to determine whether ANM regulates HNDF senescence through a SIRT-1-mediated pathway, expression levels of SIRT genes SIRT-1, SIRT-3 and SIRT-6 are is examined. In FIG. 7A, HNDFs are incubated with ANM (10 μM) or RES (5 μ) for 72 h. RT-PCR analysis indicates that SIRT-1, SIRT-3 and SIRT-6 levels are significantly increased in the ANM treatment group as compared to the control group. SIRT-1 and SIRT-3 expression levels are highly comparable to the known SIRT-1 activator resveratrol (RES). In addition, treatment with ANM also significantly increases SIRT-6, whereas a remarkable increase is observed in the RES treatment group.

In FIG. 7B, HNDFs are exposed to HG in the presence or absence of ANM (10 μM) or RES (50) for 72 h. Total RNA is extracted and subjected to Q-PCR analysis to monitor SIRT-1, SIRT-3 and SIRT-6 expression. Relative mRNA levels are normalized by β-actin mRNA. Previous studies have shown that exposure of endothelial cells to HG rapidly decreases levels of expression of SIRT genes. The results also demonstrate that exposure of HNDFs to HG markedly decreases SIRT-1 and SIRT-6 expression as compared to that of cells exposed to NG, whereas treatment with ANM rescues SIRT-1 and SIRT-6 from HG-induced depletion.

In FIG. 7C, HNDFs are incubated with ANM (10 μM) or RES (5 μM) in the presence or absence of HG (30 mM) for 72 h. Total cell lysate is extracted and subjected to western blot analysis to monitor SIRT-1, SIRT-3, SIRT-6 protein levels. Immunoblotting further confirms that ANM significantly prevented HG-induced reduction in SIRT-1, SIRT-3 and SIRT-6 proteins.

SIRT-1, a NAD+-dependent class III histone deacetylase has been shown to interact with a number of molecules including p53 and FoxO1. As shown in FIG. 4E, treatment with ANM significantly modulates the HG-induced acetylation in p53 and FoxO1. Next, to investigate whether the deacetylation activity of ANM is SIRT-1-dependent, deacetylation activity of ANM is determined under SIRT-1 silenced conditions.

In FIG. 7D and FIG. 7E, HNDFs are transfected with siRNA against SIRT-1 or control siRNA for 24 h or inhibited by SIRT-1 inhibitor EX527 (5 μM), and then treated with ANM (10 μM) or RES (50) in the presence of HG for 72 h. The protein expression levels of SIRT-1, SIRT-6, p21^{CIP1}, SMP30, p53, FoxO and acetylation of p53 and FoxO1 are determined by western blot analysis. In control siRNA (scrambled siRNA) transfected cells, ANM significantly increases SIRT-1 and SIRT-6 expression, whereas SIRT-1 (SIRT-6 excluded) is barely observed in SIRT-1 silenced cells (FIG. 7D). Moreover, in control siRNA transfected cells, the HG-induced expression of p21^{CIP1} (FIG. 7D) and the acetylation in p53 and FoxO1 (FIG. 7E) are significantly attenuated upon treatment of ANM with increased SMP30 expression (FIG. 7D), as compared with HG alone. However, treatment with ANM fails to inhibit the p21^{CIP1} expression and deacetylation in p53 and FoxO1 or upregulation of SMP30 in SIRT-1 silenced cells (FIGS. 7D&E). Furthermore, a similar effect is also observed in SIRT-1 inhibitor (EX527)-treated cells (FIGS. 7D&E).

In order to ascertain whether the protective effect of ANM is SIRT-1 dependent, the effect of ANM in SIRT-1 silenced HNDFs is investigated under HG conditions.

In FIG. 7F and FIG. 7G, under the same conditions, cellular senescence and cell proliferation are measured by SA-β-gal activity assay and tryphan blue exclusion assay, respectively. In control siRNA transfected cells, treatment with ANM significantly inhibits HG-induced senescence as assessed by SA-β-gal activity. However, in SIRT-1 siRNA transfected cells, SA-β-gal activity remains partially elevated despite the presence of ANM or RES (FIG. 7F). Indeed, as compared with the HG alone treatment group, ANM shows a significant inhibition of SA-β-gal activity although in the SIRT-1 silenced cells (FIG. 7F). Likewise, cell proliferation analysis also indicates that, in control siRNA transfected cells, the HG-induced reduction in cell number is significantly blocked by ANM, whereas partial protection is observed in SIRT-1 siRNA transfected cells (FIG. 7G). In addition, a similar effect is also observed in SIRT-1 inhibitor (EX527)-treated cells (FIG. 7G). The data strongly suggest that SIRT-1 partially contributes to the protective effects of ANM.

In FIG. 7H, HNDFs are transfected with siNrf2 or a combination of siNrf2 and siSIRT-1, and then incubated with ANM in the presence or absence of HG for 72 h. Cell proliferation is determined by tryphan blue exclusion assay. Interestingly, HG-induced reduction in cell proliferation is partially inhibited by ANM in Nrf2 knock-down cells, whereas ANM fails to rescue cell proliferation in Nrf2 and SIRT-1 knock-down cells. Furthermore, complete protection is achieved by co-treatment with ANM and NAC or RES. The data strongly suggest that ANM-mediated anti-oxidant defense and SIRT-1-mediated deacetylation activity regulates HG-induced senescence in HNDFs.

### HG-Induced SIRT-1 Degradation Prevented by ANM via Suppression of p38 MAPK and JNK1/2 Activation

To further understand the regulation of SIRT-1 by ANM, the effect of ANM on SIRT-1 activation and protein stability is examined under hyperglycemic conditions. In FIG. 7I, cells are incubated with JNK/SAPK or p38 MAPK inhibitors SP600125 (SP, 30 μM) and SB203580 (SB, 30 μM) in the presence of HG for 72 h. The protein expression levels of phos-SIRT-1 and SIRT-1 are determined by western blotting. Results are expressed as mean±S.E.M of three independent experiments. Statistical significance is set at ^{ϕ}P<0.05 as compared to NG vs. HG and *P<0.05 as compared to HG vs. samples. Previous studies have shown that hyperphosphorylation of SIRT-1 at serine 47 (Ser47) is correlated with enhanced endothelial senescence. In addition, persistent activation of JNK1/2 by multiple factors including hyperglycemia induces extensive SIRT-1 proteasome degradation followed by phosphorylation at Ser47. The present invention finds that a remarkable increase in SIRT-1 phosphorylation at Ser47 is observed after exposure to HG. However, treatment with ANM or RES significantly attenuates this effect. In FIG. 4F, JNK1/2 and p38 MAPK activity is increased by HG as indicated by increase in their phosphorylation, whereas ANM treatment significantly prevents HG-mediated JNK1/2 and p38 MAPK activation in HNDFs. Therefore, it is hypothesized that ANM-mediated suppression of JNK1/2 and p38 MAPK activation may have a functional role in the stability of SIRT-1 protein. Interestingly, suppression of JNK1/2 and p38 MAPK activity by a pharmacological inhibitor of JNK1/2 SP600125 and p38 MAPK SB203580 inhibits SIRT-1 phosphorylation and reduction in SIRT-1. The data suggest that SIRT-1 reduction is related to JNK1/2 activation.

C. elegans Protected from Oxidative Stress by ANM

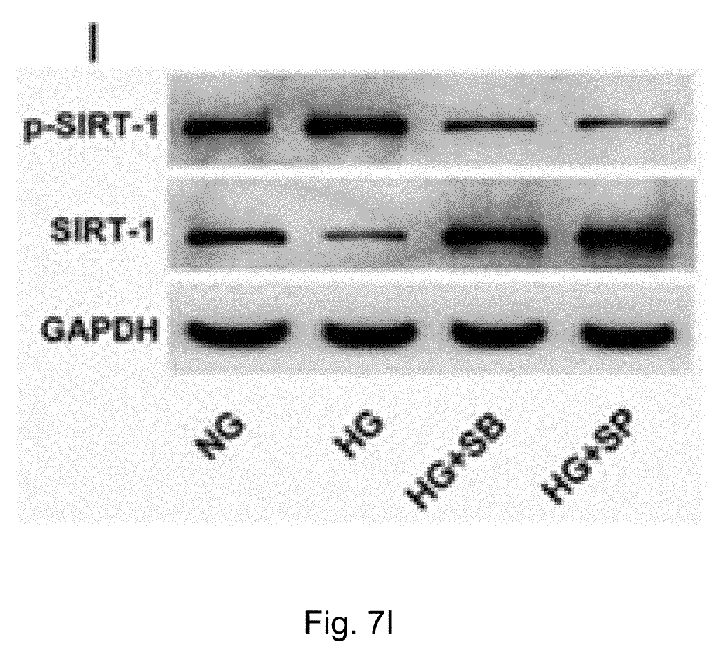

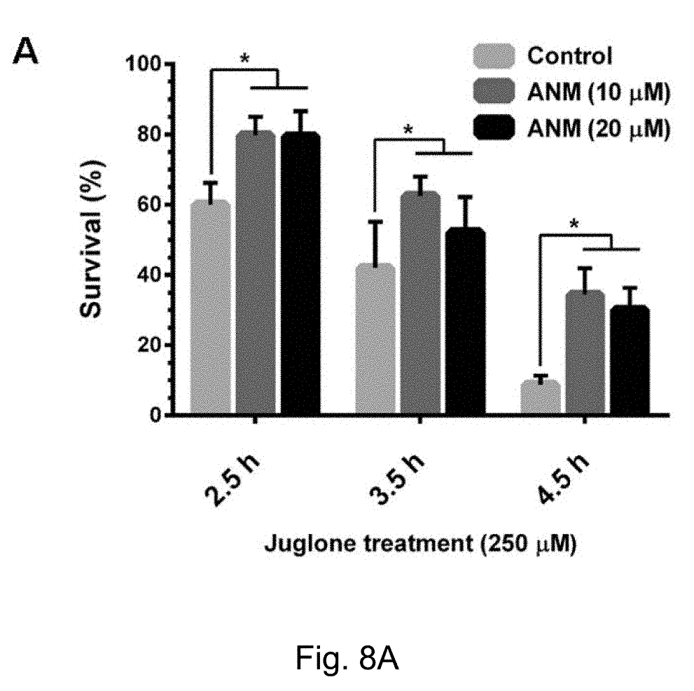

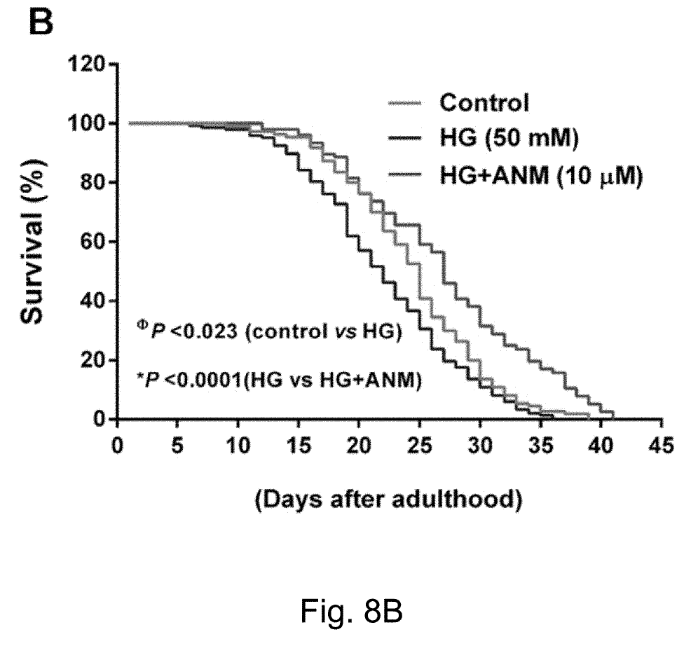

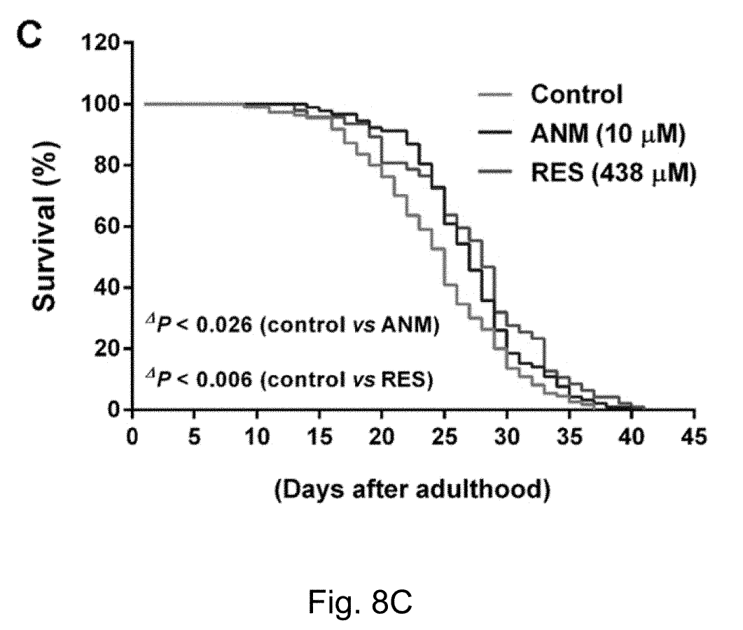

Please refer to FIG. 8A˜FIG. 8C, which are views showing the life span of wild-type C. elegans extended by ANM from oxidative stress. As shown in the figures, to further confirm the anti-oxidative property of ANM in vivo, C. elegans model is subjected. Wild-type N2 worms are pretreated with ANM for 3 days followed by exposure to oxidative stress. In FIG. 8A, to determine oxidative stress resistance, age synchronized wild-type L1 larvae are pretreated with ANM (10 and 20 μM) or DMSO (0.01%) for 3 days. Oxidative stress is induced by incubation of pre-treated worms with 250 μM Juglone for 2.5, 3.5 and 4.5 h and then scored for viability. Results are expressed as mean±S.E.M of three independent experiments. Statistical significance is ser at *P<0.05 as compared to control vs. sample treatment. The result shows that pretreatment with 10 μM ANM significantly increases the survival rate of worms exposed to oxidative stress induced by Juglone, demonstrating that ANM protects C. elegans from oxidative stress injury in vivo. It is noted that above 10 μM ANM pretreatment shows a similar effect on oxidative stress resistance to the worms.

Life Span of Wild-Type C. elegans Extended by ANM Under Hyperglycemic Condition

It has been well documented that high glucose levels decrease the life span of C. elegans by increasing ROS formation and advance glycation end-product modification of mitochondrial proteins. Therefore, it is further investigated whether ANM has a protective effect against hyperglycemia-induced oxidative stress as well as anti-aging effects. Worms are incubated with high glucose (50 mM) with or without ANM (100 μM), and controlled for life span evaluation. In FIG. 8B, effect of ANM on the life span of C. elegans under hyperglycemic condition is examined. Age synchronized L1 larvae are transfected to NGM plates which contain HG (50 mM) with or without ANM (10 μM) and worms are developed to adulthood. The survival rate is scored everyday and is expressed as a percentage of survival. In FIG. 8C, effect of ANM (10 μM) or RES (438 μM) on the life span extension of C. elegans under normal condition is examined. Results are expressed as mean±S.E.M of three independent experiments. Statistical significance is set at ^{ϕ}P<0.05 as compared to control vs. HG, *P<0.05 as compared to HG vs. HG+sample and ̂P<0.05 as compared to control vs. samples. In FIG. 8B, treatment with high glucose markedly decreases life span of C. elegans, whereas a significant (P<0.0001) increase of life span is observed in co-treatment with ANM. In addition, it is observed that ANM alone treatment significantly (P<0.026) prolongs the life span of C. elegans as compared to the control, suggesting that ANM has a protective effect against hyperglycemia-induced oxidative stress and extents life span. The effects of ANM are highly comparable with the well-known anti-aging reagent resveratrol (FIG. 8C); both compounds are originated from natural resources.

Hyperglycemia-Induced Endothelial Cells Senescence Prevented by ANM through Nrf2/SIRT-1 Activation

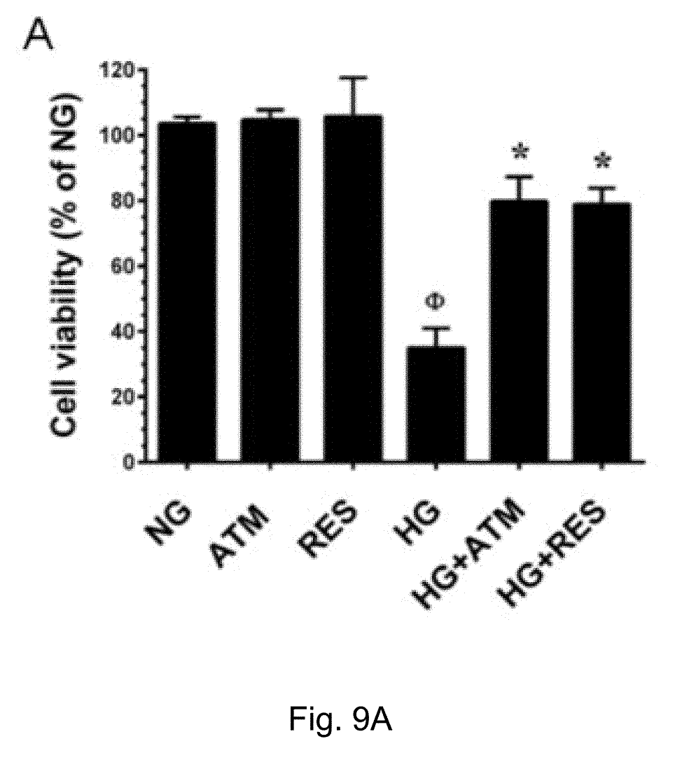

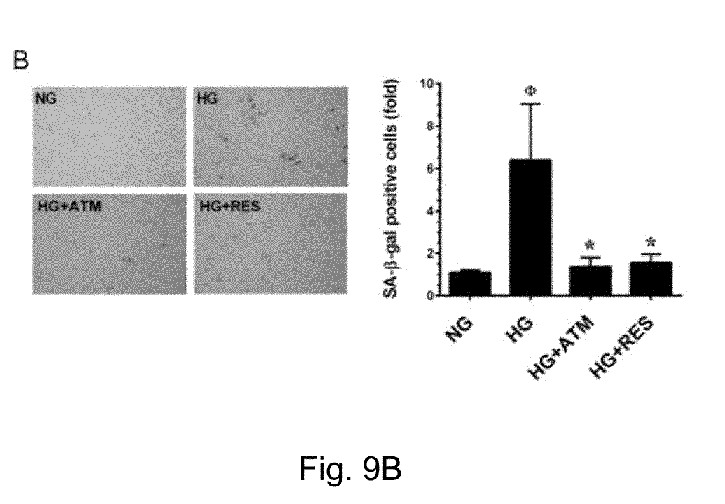

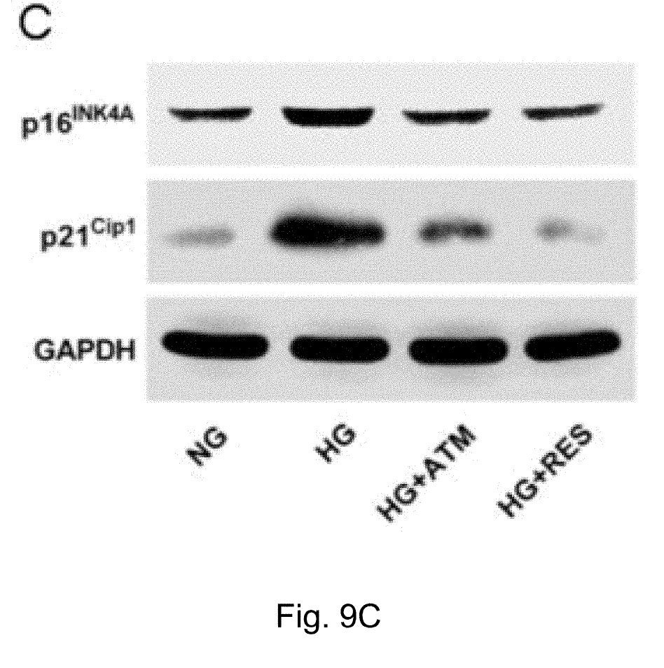

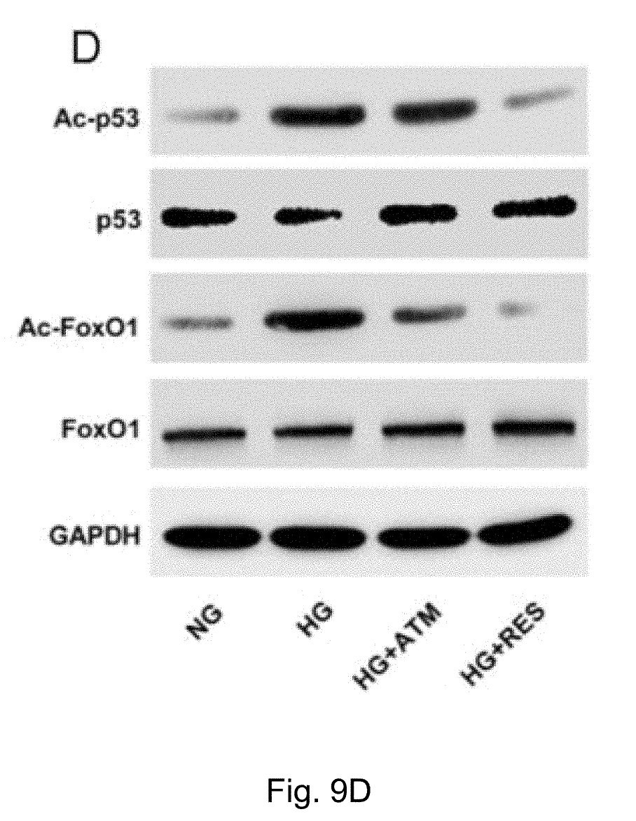

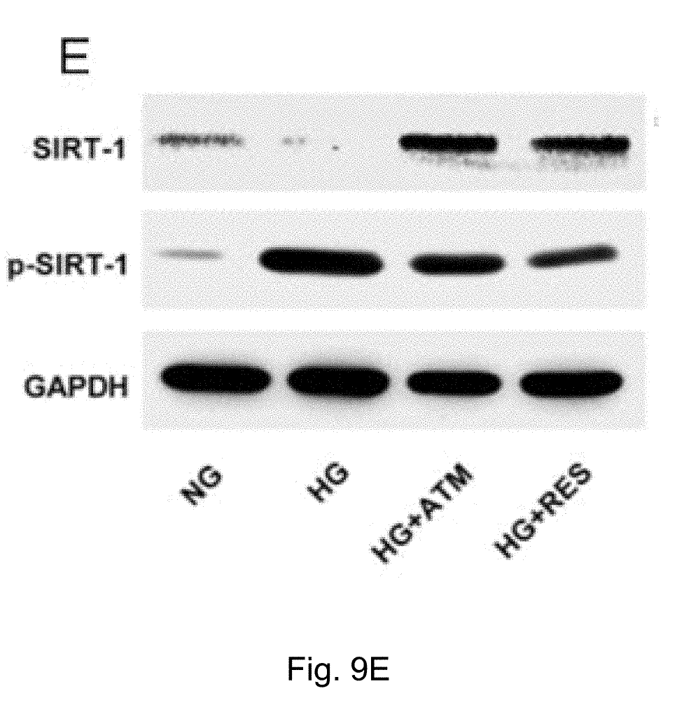

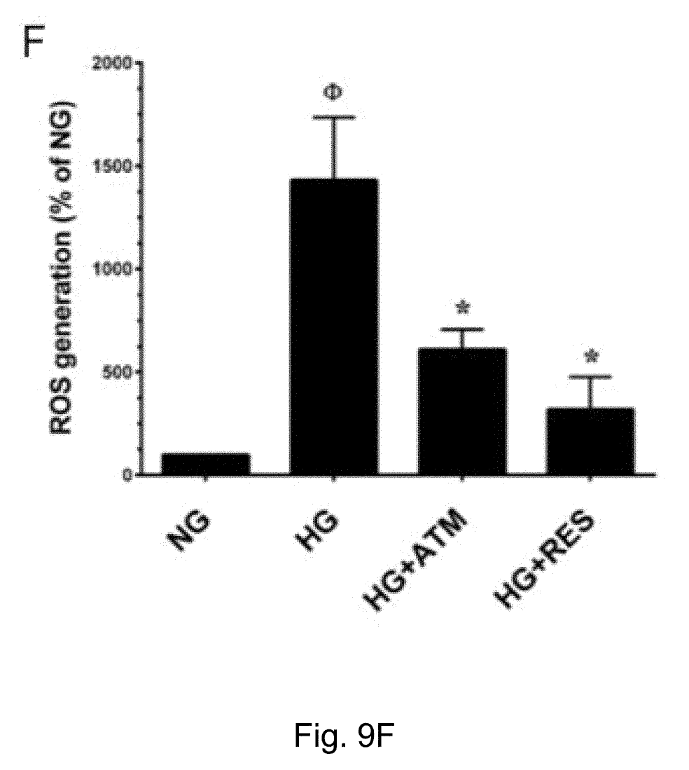

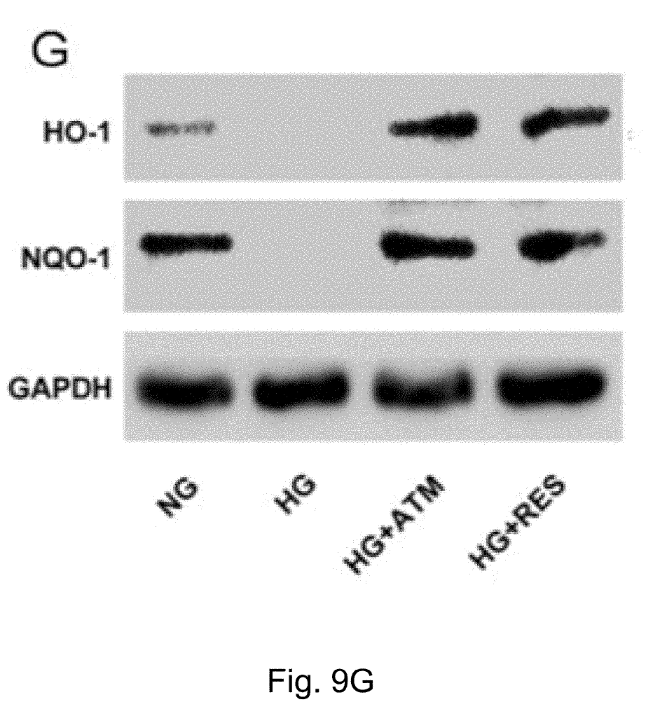

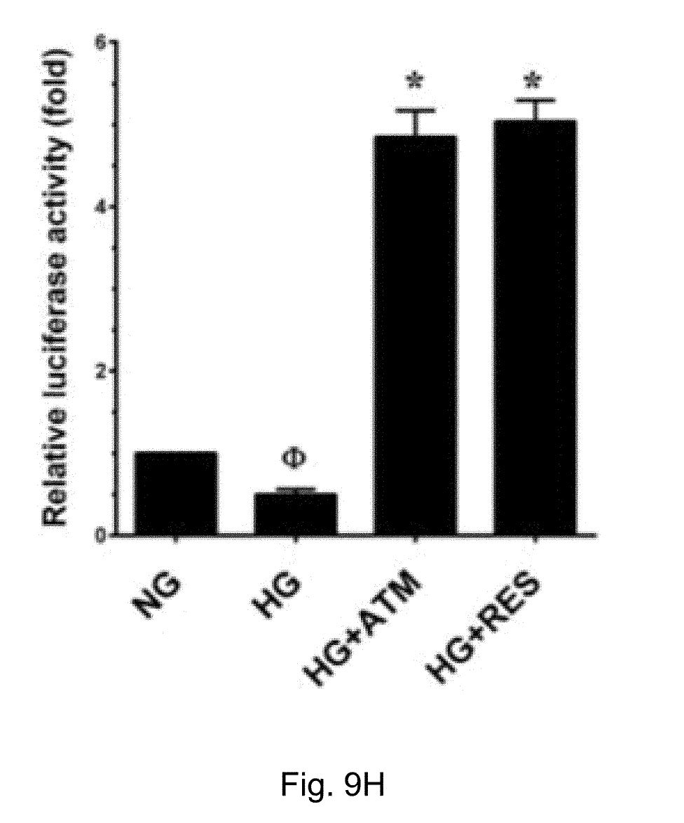

Please refer to FIG. 9A˜FIG. 9H, which are views showing HG-induced senescence prevented by ANM in human umbilical vein endothelial cells (HUVECs). As shown in the figures, to further delineate the protective effects of ANM on another cell system, experiments are designed to investigate the protective effect of ANM on HUVECs incubated in media containing either NG or HG alone or with ANM for 48 h. Cell viability is measured by MTT assay. In FIG. 9A, HUVECs are incubated with ANM (10 μM) or RES (5 μM) in the presence or absence of HG (30 mM) for 48 h. Cell viability is determined by MTT assay. Percentage of viable cells are normalized with control cells (NG). As is shown, treatment of HUVECs with ANM (10 μM) or RES (5 μM) for 48 h does not affect cell viability. However, exposure of HUVECs to HG (30 mM) for 48 h reduces number of viable cells to 35.01%, whereas co-incubation with ANM or RES significantly increases the number of viable cells to 79.78% and 78.79%, respectively. In addition, in FIG. 9B, cellular senescence is determined by SA-β-gal activity assay. SA-β-gal staining is significantly increased (6.38-fold) in HG-treated HUVECs, as compared with HUVECs maintained in NG, whereas treatment with ANM shows reduced endothelial senescence (1.36-fold), as compared with untreated HUVECs maintained in HG. In addition, in FIG. 9C and FIG. 9D, total cell lysate is extracted and senescence-associated marker proteins including p16^{INK4A}, p21^{CIP1} total and acetylated p53 and FoxO1 are measured by western blot analysis. Result from the western blot analysis also reveals that exposure of HUVECs to HG causes increased expression of p16^{INK4A} and p21^{CIP1} proteins and p53 and FoxO1 acetylation, as compared with HUVECs maintained in NG, whereas treatment with ANM significantly blocks the HG-induced p53 and FoxO1 acetylation in HUVECs.

To determine whether ANM regulates HUVEC senescence through a SIRT-1-mediated pathway, protein expression levels of SIRT-1 are examined. In FIG. 9E, protein expression levels of SIRT-1 and phos-SIRT-1 are monitored by immunoblotting. As is shown, concomitant with HNDFs, SIRT-1 levels are significantly decreased in the HG treatment group as compared to the NG, and ANM treatment significantly rescues SIRT-1 expression in HUVECs. It is also found that HG treatment markedly increases SIRT-1 phosphorylation at Ser47, whereas co-treatment with ANM significantly blocks HG-induced SIRT-1 phosphorylation. A similar effect is also observed in RES-treated cells.

In FIG. 9F, to further examine whether HG induces ROS generation which triggers endothelial senescence, HUVECs are incubated with ANM (10 μM) or RES (5 μM) in the presence or absence of HG (30 mM) for 48 h. The intracellular ROS level is quantified by utilizing DCFH-DA assay. As is shown, the production of intracellular ROS is significantly increased in HUVECs after exposure to HG (14.3-fold). However, treatment of HUVECs with ANM resulted reduced ROS levels (6.1-fold), compared with HUVECs in HG that are not treated with ANM. In addition, in FIG. 9G, western blot analysis is performed to determine the protein expression levels of HO-1 and NQO-1. Results are further confirmed through western blot analysis as compared with the NG that ANM and RES significantly increase expression of HO-1 in HG, whereas NQO-1 is unaffected by both ANM and NAC.

In FIG. 9H, HUVECs are transiently transfected with ARE promoter construct using lipofectamine and incubated with ANM (10 μM) or RES (5 μM) in the presence or absence of HG (30 mM) for 6 h. Cell lysates are mixed with luciferase reagents and quantified using an illuminometer. Relative ARE promoter activity is calculated by dividing the relative luciferase unit (RLU) of treated cells by RLU of untreated cells (NG). Results are expressed as mean±SEM of three independent experiments. Statistical significance is set at ^{ϕ}P<0.05 as compared to NG vs. HG and *P<0.05 as compared to HG vs. samples. As is shown, cell lysates activity in HUVECs transfected by ARE reporter construct significantly reduces to 0.5-fold in HG as compared to the control (1-fold). However, it is observed that the cell lysates activity is significantly increased to 4.8-fold and 5.1-fold in cells co-incubated with HG and ANM or RES, respectively.

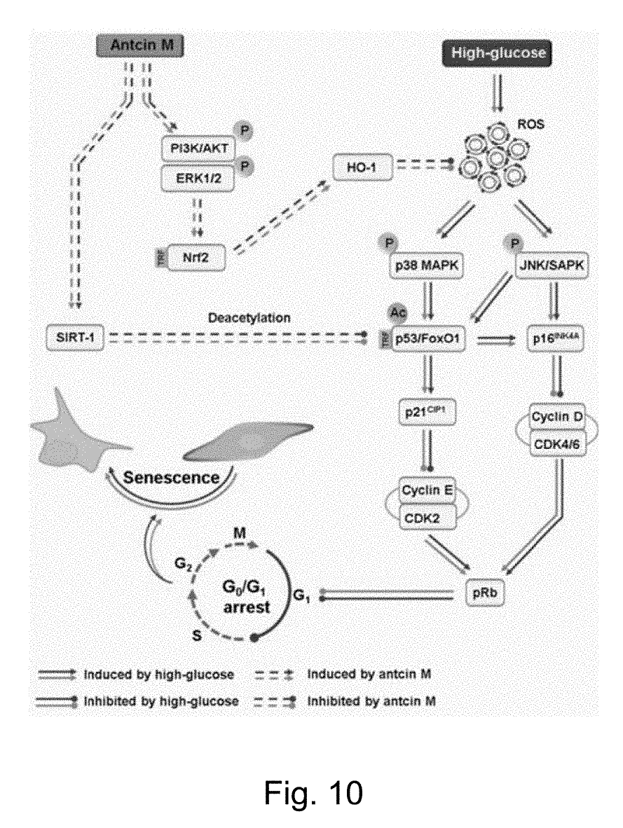

Please refer to FIG. 10, which is a view showing HNDFs and HUVECs protected from HG-stress induced premature senescence by ANM. As shown in the figure,

HG induces intracellular ROS, which triggers p38 MAPK and JNK/SAMP activation. The activated p38 MAPK and JNK/SAPK promotes transcriptional activation of p53 and FoxO1 by acetylation. P53 and FoxO1-mediated up-regulation of p16^{INK4A} and p21^{CIP1} distributes cyclins and CDKs, which increase protein stability of pRB and allow G0/G1 cell-cycle arrest and senescence. Conversely, activated p38 MAPK and JNK/SAPK reduce SIRT-1 level by phosphorylating Ser47, eventually losing deacetylation activity. However, treatment with ANM activates Nrf2-dependent anti-oxidant genes such as HO-1 and NQO-1 followed by activation of P13K/AKT and ER1/2 kinases, which facilitates ROS inhibition and upregulates SIRT-1 expression in HNDFs and HUVECs. Results are expressed as mean±SEM of three independent experiments. Statistical significance is set at ^{ϕ}P<0.05 as compared to NG vs. HG and *P<0.05 as compared to HG vs. samples.

HG induces ROS in cells and triggers activation of p38 MAPK and JNK/SAMP. The activated p38 MAPK and JNK/SAMP promote phosphorylation of p53 and FoxO1 genes and increase transcriptional activity. P53 and FoxO1 upregulate expression of p16^{INK4A} and p21^{CIP1}, hinder cyclins and CDKs in order to increase pRb stability and allow G0/G1 phase arrest and senescence. On the contrary, activated p38 MAPK and JNK/SAMP decrease expression of SIRT-1 through phosphorylation at Ser47 and lose phosphorylation activity in the end. However, ANM-treated Nrf2-dependent anti-oxidant genes including HO-1 and NQO-1 are activated followed by activation of P13K/AKT and ERK1/2 kinases, which contributes to inhibit ROS generation in HNDFs and HUVECs and upregulate SIRT-1.

### Method of Treating Aging of Cells by Administering Antcin M

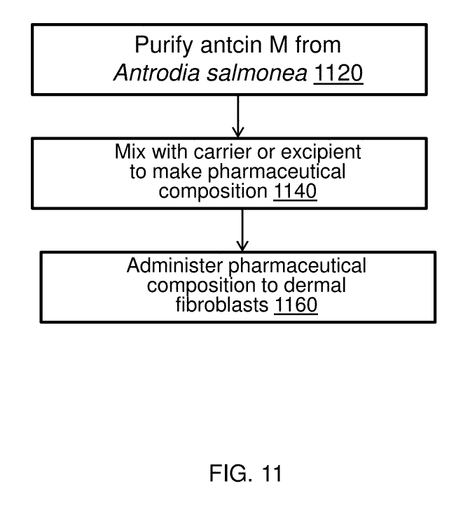

Please refer to FIG. 11 for a method of administering antcin M as a pharmaceutical composition to treat the aging of cells. In the method, the antcin M (ANM) is isolated as discussed in the materials and methods from Antrodia salmonea (see step 1120 in FIG. 11). In some embodiments, the antcin M is isolated from the fruiting bodies of Antrodia salmonea. In some embodiments, the antcin M is isolated to remove cytotoxic concentrations of antcin B and/or antcin K. In other embodiments, other cytotoxic antcins can be removed. In other embodiments, the amount of antcin B and/or antcin K that is removed is enough to render the pharmaceutical composition nontoxic to cells. In some embodiments, the antcin M is mixed with other noncytotoxic antcins (e.g., antcin A, antcin C, antcin H). In some embodiments, the purity of the antcin M is 99%.

In the second step (see 1140 in FIG. 11) antcin M is admixed with a pharmaceutically acceptable carrier or excipient. The pharmaceutically acceptable carrier or excipient can be chosen based on the type of administration and can include, but is not limited to, a topical carrier or excipient, an intravenous, intradermal, oral, or subcutaneous carrier or excipient.

The third step involves administering the pharmaceutical composition to cells in an amount effective to reduce cellular aging or senescence. In at least one embodiment, the pharmaceutical composition is administered in any way that allows treatment of affected dermal fibroblasts and/or endothelial cells. In some embodiments, the pharmaceutical composition is administered topically to the epidermis of a human to treat skin aging and the pharmaceutically acceptable carrier or excipient is a lotion or other substance that allows for ease of application. In at least one embodiment, the pharmaceutical composition also includes some type of sunscreen agent to reduce UV exposure.

In at least one embodiment, the method is used to treat cellular aging due to diabetes mellitis, including impaired wound healing, ulceration, and/or skin aging. In this embodiment, the pharmaceutical composition can be administered topically, intradermally, subcutaneously, and/or intravenously to treat dermal fibroblasts and endothelial cells. In other embodiments, the pharmaceutical composition is administered via any method that allows treatment of dermal fibroblasts and endothelial cells that are affected by diabetes mellitis, including, but not limited to, topically, intravenously, subcutaneously, orally, and/or intradermally.

In at least one embodiment, the pharmaceutical composition is used to treat wound healing in diabetes mellitis patients.

The amount that is effective to treat cellular aging or senescence may vary depending on the type of administration. However, the amount may be an amount effective to prevent hyperglycemia-induced G₀/G₁ phase arrest and senescence. In other embodiments, the amount prevents hyperglycemia-induced oxidative damage. In other embodiments, the amount activates NF-E2 related factor-2 (Nrf2)-mediated anti-oxidant genes and eliminates HG-induced reactive oxygen species (ROS). In other embodiments, the effective amount abolishes or reduces stress-induced premature senescence (SIPS) in the presence of HG by reducing senescence-associated β-galactosidase (SA-β-gal) activity in dermal fibroblasts. In other embodiments, the effective amount increases expression of silent mating type information regulation 2 homologs 1 (SIRT-1) in dermal fibroblasts.

In other embodiments, the effective amount is a concentration from about 10 μA to about 30 μNA, including but not limited to: 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, and 29 μM. In other embodiments, the effective amount is from about 15 μM to about 25 μM.

### Discussion

Cellular senescence is an inevitable process by which cells irreversibly exit the cell-cycle and stop dividing in response to a variety of stresses including those observed during hyperglycemic states. In the present invention, human premature skin aging in vitro is modeled by culturing normal human dermal fibroblasts (HNDFs) with high-glucose (30 mM) to investigate the protective role of ANM in cell senescence.

New therapeutic agents from natural sources have potential pharmacological properties for complicated human diseases such as diabetes and aging. Several phytochemicals including phenolic compounds, flavonoids and terpenoids exhibit anti-diabetic and anti-aging properties through their anti-oxidant or anti-inflammatory effects. Antcins (ANA, ANB, ANC, ANH, ANK and ANM) are naturally occurring triterpenoids reported to have anti-oxidant and anti-inflammatory effects; therefore, they might have beneficial effects on diabetic mellitus and aging. Initial cytotoxic assessment shows that ANA, ANC, ANH and ANM are not cytotoxic to the HNDFs at high concentrations (20 μM). However, ANB and ANK are highly toxic to the HNDFs, which agrees with previous studies where ANB and ANK are cytotoxic to the hepatoma cell line. Therefore, next, the anti-aging effect of ANA, ANH and ANM is examined. Stress-induced premature senescence (SIPS) is evident in fibroblasts incubated in hyperglycemic (>25 mM) medium, as compared to those cultured in medium containing physiological concentration of glucose (5.5 mM), confirming the paradoxical relationship between glucose concentration and SIPS by observation of various characteristic features of cellular senescence. Senescence-associated β-galactosidase (SA-β-gal) activity is significantly increased by hyperglycemia, suggesting that cells experience senescence, whereas the hyperglycemia-induced increase in SA-β-gal positive cells is significantly inhibited by ANA, ANH and ANM. However, a highly pronounced inhibitory effect is observed in ANM-treated cells. Immunoblotting shows that ANA, ANH and ANM significantly down-regulated the HG-induced increase in p16^{INK4A}, a tumor suppressor protein known to transduce senescence-signals and lead to irreversible growth arrest. In addition, ANA, ANH and ANM significantly inhibit HG-induced p21^{CIP1} expression, a cyclin-dependent kinase inhibitor that regulates growth-arrest and cellular senescence. Regucalcin, also known as SMP30, is a 34-kDa cytosolic marker protein of cellular aging, which rapidly losses its expression during senescence. The present invention shows a significant decrease in the levels of SMP30 in HNDFc that had been cultured in HG for 72 h, whereas in the presence of antcins, restores SMP30 expression. Taken together, the present invention suggests that, as compared to other antcins, ANM exerts a potent beneficial effect on hyperglycemia-induced senescence through modulating the p16^{INK4A} and p21^{CIP1} pathways.

The results of the present invention and previous studies, indicate that glucose at a concentration of 30 mM is sufficient to induce SIPS onset in dermal fibroblasts. Although, HG enhances ROS production, which causes oxidative damage and chronic ailments including diabetes. In the present invention, it is found that HNDFs exposed to HG exhibits characteristics associated with aging via increased ROS production and SA-β-gal activity. However, co-incubation with ANM robustly attenuates ROS generation and SA-β-gal activity. The relevant role of oxidative stress in senescence is demonstrated by the fact that treatment with anti-oxidants delays or eliminates cellular senescence. Mechanically, excessive intracellular ROS levels lead to increased transcriptional activity of p53 through the acetylation at Lys382, which eventually up-regulates p21^{CIP1}. The human endothelial cells cultured in hyperglycemic medium shows marked SA-β-gal activity in association with increased DNA damage markers, p16^{INK4A} p21^{CIP1} and p53. The present invention is evidence that exposure of HNDFs to HG for 72 h increases the expression of p21^{CIP1} protein and increases p53 acetylation. This is the first report indicating HG-induced p53 acetylation in dermal fibroblasts. This finding is in accordance with a previous study that reported hyperglycemia accelerates p53 acetylation through intracellular ROS accumulation. Therefore, the hypothesis that ANM would inhibit HG-induced p53 acetylation in HNDFs is examined. Data from the present invention demonstrate that co-treatment with ANM significantly attenuates HG-induced p53 acetylation and decreases the expression of p21^{CIP1} protein. In parallel, HNDFs cultured in HG show a significant increase in FoxO1 acetylation. The increase in FoxO1 acetylation may result in the transcriptional activation of FoxO1 towards the transcription of cell-cycle arrest genes, which are stimulated with HG-induced oxidative stress. However, the presence of ANM significantly attenuates the FoxO1 acetylation in HNDFs exposed to HG. In addition, phosphorylation of FoxO1 at Thr24 by AKT promotes cell survival by regulating cell-cycle progression. Data from the present invention also show that HG treatment causes a remarkable decrease in FoxO1 phosphorylation, and in the presence of ANM, HG fails to abrogate FoxO1 phosphorylation in HNDFs. These data support the hypothesis that ANM provokes HG-induced senescence through the negative regulation of p53 and FoxO1. This is the first report indicating HG-induced p53 and FoxO1 acetylation in dermal fibroblasts. In addition, it is well demonstrated that activation of JNK1/2 by ROS triggers p53 activation. However, this pathway involved in premature senescence is poorly elucidated. In the present invention, an aberrant activation of JNK1/2 is found in HG-treated cells, whereas the JNK1/2 phosphorylation is barely observed in ANM and NAC, and ROS inhibitor treated cells. These data suggest that HG-induced ROS might trigger JNK1/2 activation, which may lead to p53 activation and premature senescence.

The number of stimuli known to induce SIPS is constantly increasing and the mechanism has been extensively studied. Increased senescence has been shown to be associated with the expression of p16^{INK4A} protein in endothelial cells cultured in hyperglycemic medium, this effect is blocked by stachydrin, a proline betaine found in citrus juice. Data from the present invention show that HG-induced increase of p16^{INK4A} significantly blocks by ANM. Several reports have shown that the ability of ROS to induce p16INK4 depends on p53 activation via its upstream kinase p38 MAPK. Constitutive activation of this pathway induces p16^{INK4A} and p21^{CIP1} and leads to premature senescence. Robust activation of p38 MAPK is observed in HG-treated cells, and this activation is significantly blocked by ANM. This result suggests that ANM exerts a beneficial effect on HG-induced senescence through modulating the p16^{INK4A} and p38 MAPK cascades. Likewise, intracellular ROS activates JNK/SAPK, which triggers p53 transcriptional activity. However, the link between JNK/SAPK and p16^{INK4A} remains unknown. In the present invention, it is found that inhibition of JNK/SAPK activity by pharmacological inhibitor results in reduced p16^{INK4A} protein and p53 activation, as compared to cells that are treated only in HG. The present invention is the first data demonstrating the link between JNK/SAPK and p16^{INK4A}.

Cell-cycle arrest and senescence is a frequently discussed topic in aging-related research. Blagosklonny extensively reviewed the difference between quiescence and senescence. Quiescent cells are capable of restarting proliferation by addition of growth factors. Nevertheless, senescent cells arrest at G0/G1 phase and are unable to restart proliferation. In line with the previous studies, HG arrests cells in G1-S transition phase, and increases cell population in the G0/G1 phase. This effect is blocked by co-treatment with ANM which keeps the percentage of cells in the G0/G1 phase near to control values. Cell-cycle progression is regulated by complexes of cyclins and cyclin-dependent kinases (CDKs), and reduction in the complex of cyclin D with CDK4/CDK6 and cyclin E with CDK2 results in G1-S transition arrest. In addition, disruption of cyclin/CDK complex promotes retinoblastoma protein (pRb) stability and prevents the progression from the G1 to S phase of the cell division via inhibiting the transcription factor E2F family which plays a major role in G1-S transition in mammalian cells. In the present invention, it is found that treatment with HG results in decreased pRb phosphorylation followed by reduction in cyclin D1, CDK4, CDK4, cyclin E and CDK2, which is eliminated following co-treatment with ANM. The results obtained from cell-cycle analysis are consistent with this observation.

Eukaryotic cells are fortified with primary and secondary defense against oxidative stress insults. Particularly, the phase II enzymes such as hemeoxygenase-1 (HO-1), NAD(P)H:quinone oxidoreductase 1 (NQO1), and glutathione-S-transferase (GST) are rapidly activated by an endogenous mechanism through which oxidative toxicants can be removed before they damage DNA. Many natural products have been reported to have beneficial effects on the aging processes: polyphenols, flavonoids, terpenoids, caratinoids, vitamins, resveratrol, curcumin, ferulic acid and caffeic acid, are well-known for their high anti-oxidant content. These components act not only as free radical scavengers but also by modulating signal transduction pathways and gene expression patterns. In the present invention, ANM shows strong inhibition of HG-induced ROS generation, which demonstrates the anti-oxidant efficacy of ANM. Further analysis reveals that ANM does not have a direct free-radical scavenging effect as measured by DPPH assay. A previous study showed that ANC, a similar analog of ANM, exerts free-radical-induced oxidative stress in hepatocytes through the induction of Nrf2-dependent anti-oxidant genes. However, ANM eliminates excessive ROS generation through the induction of anti-oxidant genes such as HO-1 and NQO-1. The increased levels of anti-oxidant genes are observed after co-treatment with HG and ANM. Data from the present invention suggests that ANM induces anti-oxidant genes upon excessive oxidative stress. In contrast, treatment with HG also increases HO-1 and NQO-1 mRNA levels. However, the NQO-1 expression under HG is not statistically significant with control cells. Nrf2, a bZIP transcription factor, regulates the expression of anti-oxidant genes including HO-1 and NQO-1. Under normal physiological conditions, Nrf2 is sequestrated in the cytoplasm, and, upon stimulation, disassociates from its cytosolic inhibitor Keap-1, translocates into the nucleus and binds to the cis-acting anti-oxidant responsible element (ARE) in the promoter region. Many studies have shown that ARE promoter is targeted by dietary phytochemicals as evidenced by the finding that deletion of ARE-site containing E1 and E2 regions blunts induction. In the present invention, it is demonstrated that treatment with ANM significantly increases the transcriptional activity of Nrf2 in HG-induced HNDFs.

Senescence-related hyperglycemia is associated with increased oxidative stress via MAPKs. Moreover, the transcription factor Nrf2 is activated by upstream kinases including PI3K/AKT, PKC, JNK/SAPK, ERK1/2 and p38 MAPK. In the present invention, PI3K/AKT and ERK1/2 are significantly up-regulated by ANM under normal and hyperglycemic conditions, which may be associated with Nrf2 activation. Result shows that ANM-induced Nrf2 transcriptional activity is significantly abolished by P13K/AKT and ERK1/2 inhibitors, which demonstrates that ANM-induced Nrf2 activity is mediated by the P13K/AKT and ERK1/2 cascades. Indeed, a remarkable increase in Nrf2 activity is observed in JNK/SAPK inhibitor treated cells supporting the notion that JNK/SAPK downregulates Nrf2 activity in HG-treated cells. Data from the present invention is consistent with previous study that ANC induces Nrf2 activity via activation of the P13K/AKT and JNK/SAPK pathways. Moreover, the activation of Nrf2 by phytochemicals is involved in various upstream mechanisms. For example, curcumin, caffeic acid and suphoraphane directly target the thiol group of Keap-1 to induce proteasomal degradation, which promotes Nrf2 transcriptional activity. Furthermore, silencing Nrf2 by siRNA fails to protect HG-induced cellular senescence even in the presence of ANM, demonstrating the role of Nrf2-mediated anti-oxidant mechanism in oxidative stress-induced premature senescence.

A growing body of evidence suggests that SIRT-1 is an important modulator of cellular senescence, longevity, metabolism and apoptosis. Previous studies show that inhibition of SIRT-1 by sirtinol or SIRT-1 siRNA results in a premature senescence-like phenotype in endothelial and young mesenchymal stem cells and overexpression of SIRT-1 reverses this processes. In addition, hyperglycemia accelerates endothelial cell senescence which is associated with reduction in SIRT-1. These studies imply that SIRT-1 plays a pivotal role in regulation of cellular senescence. In the present invention, it is found that treatment with ANM alone could increase SIRT-1 mRNA expression along with SIRT-3 and SIRT-6. In line with a previous study, exposure of HNDFs to HG causes a dramatic reduction in SIRT-1 and SIRT-6 mRNA and protein expression levels. However, co-incubation with ANM counteracts the detrimental effects of HG by upregulating SIRT-1 and SIRT-6 expression levels. It has been reported that hyper-phosphorylation of SIRT-1 at Ser47 by JNK/SAPK induces proteasome degradation of SIRT-1 in fibroblasts. However, other factors involved in SIRT-1 phosphorylation at Ser47 are poorly understood. Interestingly, data from the present invention show that treatment with HG increases JNK/SAPK activation as well as SIRT-1 phosphorylation in HNDFs. This connection is further confirmed by observation of very little HG-induced SIRT-1 depletion and SIRT-1 phosphorylation in JNK/SAPK inhibitor-treated cells. A similar effect is also observed in p38 MAPK inhibitor-treated cells. These data confirm that HG-induced SIRT-1 depletion is coordinated by JNK/SAPK and p38 MAPK via increased SIRT-1 hyper-phosphorylation and proteasome degradation. Previous studies suggested that the protective effect of SIRT-1 may be due to the regulation of acetylation/deacetylation of key transcription factors such as p53 and FoxO1. Activation of p53 by external or internal stimuli induces expression of several genes including p21^{CIP1} and P16^{INK4A}, which are bound to the G1-S transition kinases (CDK4, CDK6, CDK2 and CDK1) and inhibit their activity. Likewise, FoxO1 transcription factor plays a crucial role in cellular senescence by upregulating p21^{CIP1} and p16^{INK4A} genes. In the present invention, HNDFs exposed to HG shows a significant decrease in SIRT-1 expression and a parallel significant increase in p53 and FoxO1 acetylation. This increase in p53 and FoxO1 acetylation may result in the switching of p53 and FoxO1 transcriptional activity towards transcription of growth inhibition or senescence inducible genes. The presence of ANM significantly reduces p53 and FoxO1 acetylation with a subsequent decrease in p21^{CIP1} and p16^{INK4A}. Data from the present invention also show that treatment with ANM does not significantly attenuate the p53 and FoxO1 acetylation and p21^{CIP1} and p16^{INK4A} expression in SIRT-1-silenced HNDFs exposed to HG. Surprisingly, treatment with ANM partially protects HG-induced cellular senescence and cell survival in SIRT-1 knock-down cells, which further suggests that the protective effect of ANM is done through its anti-oxidative properties. This is confirmed by the fact that ANM fails to protect HG-induced senescence and growth arrest in SIRT-1 and Nrf2 knock-down cells. Similarly, a synergistic effect is observed when ANM is combined with a well-known antioxidant NAC and SIRT-1 enhancer resveratrol.

To further understand the effects of ANM in vivo, C. elegans is used as an in vivo model to examine the protective and anti-aging effects of ANM. There are number of studies demonstrate that the protective actions of phytochemicals in C. elegans are mainly attributed to their antioxidative potential. The present invention shows that the survival rate of wild-type worms are significantly increased with ANM treatment under Juglone-induced oxidative stress condition, suggesting that ANM has strong antioxidative activity in vivo. Schulz et al reported that C. elegans raised under HG condition lost the ability to oxidize glucose and suffered reduced fertility and decreased total progeny production. In addition, glucose enriched diet had significantly decreased C. elegans life span due to increased ROS formation. It is also found a reduction in the life span under a high glucose condition, whereas co-treatment with ANM significantly increases life span, suggesting that ANM has a protective effect against HG-induced oxidative stress.

Next to investigate whether the protective effect of ANM is limited to fibroblasts or extends to other organs, hyperglycemia-induced endothelial senescence and the protective effect of ANM are examined. Interestingly, ANM shows a similar protective effect against HG-induced endothelial senescence. Taken together, data from the present invention thus support the hypothesis that ANM promotes anti-oxidant defense and SIRT-1 stability in hyperglycemia-induced dermal fibroblasts and endothelial cells that minimize cellular senescence and growth arrest (FIG. 10). Further in vivo studies demonstrate that ANM is a novel anti-aging reagent that confers an increase in oxidative stress resistance and extends life span on the nematode C. elegans.

### Materials and Methods

### I. Chemicals and Reagents

ANA, ANB, ANC, ANH, ANK and ANM are isolated from the fruiting bodies of A. cinnamomea and A. salmonea as described previously. The purity of the antcins is above 99% as confirmed by HPLC and FT-NMR analysis. Minimum essential medium (MEM), Medium 199 (M-199), fetal bovine serum (FBS), sodium pyruvate, penicillin and streptomycin are obtained from Invitrogen (Carlsbad, Calif.). Heparin sodium salt, endothelial cell growth supplement (ECGS), N-acetylcysteine, 2′,7′-dichlorofluorescein diacetate (DCFH2-DA), 3-(4,5-dimethyl-thiazol-2-yl)-2,5-diphenyl tetrazolium bromide (MTT) and D-Glucose are purchased from Sigma-Aldrich (St Louis, Calif.). Antibodies against cyclin D1, cyclin B1, cyclin E, CDK2, CDK4, CDK6, Cdc2, phos-pRb, p16^{INK4A}, p21^{CIP1} acety-p52, phos-p53, phos-FoxO1, FoxO1, phos-JNK/SAPK, JNK/SAPK, phos-p38 MAPK, p38 MAPK, phos-ERK1/2, ERK1/2, Phos-AKT, AKT, histone H3, phos-SIRT-1, SIRT-1, SIRT-3, SIRT-6 and Keap-1 are obtained from Cell Signaling Technology, Danvers, Mass. Antibodies against p53, SMP30 and acetyl-FoxO1 are purchased from Santa Cruz Biotechnology, Dallas, Tex. Antibodies against HO-1 and NQO-1 are obtained from Abcam, Cambridge, UK. All other chemicals are reagent grade or HPLC grade and supplied by either Merck (Darmstadt, Germany) or Sigma-Aldrich.

### II. Method

A. Cell Culture and Sample Treatment

HNDFs (CCD966SK) and human umbilical vein endothelial cells (HUVECs) are obtained from the Bioresource Collection and Research Center (BCRC), Hsinchu, Taiwan. HNDFs are grown in MEM containing 10% FBS, 2 mM L-glutamine, 100 U/mL penicillin and streptomycin at 37° C. in a fully humidified atmosphere of 5% CO2. Likewise, HUVECs are grown in M-199 medium supplemented with ECGS, heparin, 10% FBS and 100 U/mL penicillin and streptomycin at 37° C. in a fully humidified atmosphere of 5% CO2. High-glucose treatment is performed by treating cells with 15 or 30 mM D-glucose (HG) for 24-72 h. HNDFs and HUVECs are also treated with HG in the presence of 10 μM ANM or 100 μM N-acetylcysteine or 50 resveratrol. Controls are performed in the presence of media with normal glucose alone (NG, 5.5 mM) or with 10 μM ANM or 100 μM N-acetylcysteine or 5 μM resveratrol.

B. Cell Viability and Proliferation Assay

Cell viability is assessed by MTT colorimetric assay. Briefly, HNDFs (2×10⁴cells/well) or HUVECs (5×10⁴ cells/well) are seeded in a 24-well culture plate. After treatment with HG (15 and 30 mM) in the presence or absence of samples for 24-72 h, culture media is withdrawn and incubated with MTT (1 mg/mL) in fresh medium for 2 h. The MTT formazan crystals are dissolved in 400 μL of DMSO and the samples are measured at 570 nm (A540) using an ELISA microplate reader (Bio-Tek Instruments, Winooski, Vt.). The percentage of cell viability (%) is calculated as (A570 of treated cells/A570 of untreated cells)×100.

Cell proliferation is evaluated using trypan blue exclusion assay as described previously with minor modification. Cells are plated into 6-well plates at a density of 5×10⁴ cells/well. After incubation overnight, cells are treated with test samples in the presence or absence of HG for 24-72 h. Cells exposed to 0.2% Trypan blue are then counted in a hemocytometer, and cells stained with Trypan blue are excluded. Percentage of viable cells is calculated based on the ratio of viable cells to total cell population in each well. The proliferation rate is calculated based on the number of viable cells in HG or sample-treated groups versus the NG-treated group.

C. Apoptosis Assay

The assay of Annexin V and PI binding staining is performed with an Annexin V-FITC/PI Apoptosis Detection Kit according to the manufacturer's instructions (BD Bioscences, San Jose, Calif.). Briefly, 5×10⁵ cells/dish are seeded in a 10 cm culture dish, after incubation overnight, cells are exposed to HG (15-30 mM) or NG (5.5 mM) for 72 h. Cells are washed twice with PBS and collected using 0.25% trypsin without EDTA, cells are pooled by centrifuging at 1500×g for 5 min. Then, cells are suspended in 500 μL of binding buffer which contained 1 μL Annexin V-FITC and 5 μL PI and incubated with the cells for 5 min in the dark. The stained cells are analyzed directly by flow cytometer (Beckman Coulter, Brea, Calif.). Data are acquired and analyzed using CXP software (Beckman Coulter).

D. Cell-Cycle Analysis

HNDFs at a density of 5×10⁵ cells in 10 cm dishes are treated with ANM or NAC in the presence or absence of HG for 72 h. Cells are collected, washed with PBS and fixed in 95% cold-ethanol, and kept at −20° C. overnight. The cell pellet is then washed again with PBS and centrifuged at 1500×g for 5 min. The pellet is re-suspended in 1 mL PI/Triton X-100 (20 μg/mL PI, 0.1% Triton X-100 and 0.2 mg/mL RNAse) and incubated on ice for 30 min. The total cellular DNA content is analyzed with a flow cytometer (Beckman Coulter FC500). Data are acquired and analyzed using CXP software (Beckman Coulter).

E. Flow Cytometric Detection of Intracellular ROS

Intracellular ROS accumulation is determined using the dye DCFH2-DA following a procedure described earlier. Briefly, HNDFs (1×10⁵ cells/well) are seeded in 6-well plates and incubated with HG in the presence or absence of test samples for 24 h. At the end of the incubation, the culture supernatant is removed and cells are washed twice with PBS. DCFH2-DA (100) is mixed with 500 μL MEM and added to the culture plate. After incubation for 30 minutes, cells are collected by trypsin and the fluorescence shift is quantified using a flow cytometer (Beckman Coulter). Data are acquired and analyzed using CXP software (Beckman Coulter).

F. Senescence-Associated β-galactosidase Activity Assay

Senescence-associated β-galactosidase (SA-β-gal) activity is determined in formaldehyde-fixed histochemical staining kit according to the manufacturer's instructions (Cell Signaling Technology, Danvers, Calif.). Briefly, cells are grown in 6-well plates at a density of 5×10⁴ cells/well, and incubated with HG or test samples for 48 h (HUVECs) or 72 h (HNDFs). After incubation, cells are stained with SA-β-gal staining solution at pH 6.0 overnight and then the development of blue staining is observed and photographed under a bright-field microscope (Motic Electric Group, Xiamen, P.R. China).

G. Immunofluorescence

HNDFs at a density of 1×10⁴ cells/well are cultured in an eight-well glass Nunc Lab-Tek chamber (ThermoFisher Scientific, Waltham, Mass.). Cells are treated with ANM or NAC in the presence or absence of HG for 2-72 h. After incubation, culture medium is removed and cells are fixed in 4% paraformaldehyde for 15 min, permeabilized with 0.1% Triton X-100 for 10 min, washed and blocked with 10% FBS in PBS, and then incubated overnight with the corresponding primary antibodies in 1.5% FBS. The cells are then incubated with the fluorescein isothiocyanate (FITC)-conjugated secondary antibody (Alexa fluor 488, ThermoFisher Scientific) for another 1 h in 6% bovine serum albumin (BSA). Next, the cells are stained with 1 μg/mL 4′,6-diamidino-2-phenylindole (DAPI, Cell Signaling Technology) for 5 min, washed with PBS, and visualized using a fluorescence microscope (Motic Electric Group) at 40× magnification.

H. RNA Extraction and Q-PCR Analysis

Total RNA is extracted from cultured HNDFs using Trizol Reagent (Thermo Fisher Scientific). Q-PCR analysis is performed using Applied Biosystems detection instruments and software. Forward and reverse primers (10 μM), and the working solution SYBR green, is used as a PCR master mix, under the following conditions: 96° C. for 3 minutes followed by 40 cycles at 96° C. for 1 minute, 50° C. for 30 seconds and 72° C. for 90 seconds. GAPDH is used as an internal standard to control for variability in amplification because of differences in starting mRNA concentrations. The copy number of each transcript is calculated as the relative copy number normalized by GAPDH copy number. The sequences of the PCR primers are as summarized in Table 1.

TABLE 1

| Gene | Sequence |
|---|---|
| SIRT-1 | Forward: 5′-GCAGATTAGTAGGCGGCTTG |
| | Reverse: 5′-TCTGGCATGTCCCACTATCA |
| SIRT-3 | Forward: 5′-CATGAGCTGCAGTGACTGGT |
| | Reverse: 5′-GAGCTTGCCGTTCAACTAGG |
| SIRT-6 | Forward: 5′-AGGATGTCGGTGAATTACGC |
| | Reverse: 5′-AAAGGTGGTGTCGAACTTGG |
| HO-1 | Forward: 5′-TCAACGGCACAGTCAAGG-3′ |
| | Reverse: 5′-ACTCCACGACANACTCAGC-3′ |
| NQO-1 | Forward: 5′-TGCGGTGCAGCTCTTCTG-3′ |
| | Reverse: 5′-GCAACCCGACAGCATGC-3′ |
| β-actin | Forward: 5′-TCAACGGCACAGTCAAGG-3′ |
| | Reverse: 5′-ACTCCACGACANACTCAGC-3′ |

I. Protein Extraction and Western Blot Analysis

HNDFs or HUVECs (1×10⁶ cells/dish) are cultured in 10-cm dishes and treated with ANM or NAC or RES in the presence or absence of HG for 48-72 h. Cells are lysed by either RIPA lysis buffer or nuclear and cytoplasmic extraction reagents (Thermo Fisher Scientific). Protein concentrations are determined by Bio-Rad protein assay reagent (Bio-Rad Laboratories, Hercules, Calif.). Equal amounts of protein samples (60 μg) are separated by 7-12% SDS-PAGE and the separated proteins are transferred onto polyvinylidene chloride (PVDC) membrane overnight. The transferred protein membranes are blocked with 5% non-fat dried milk for 30 min, followed by incubation with specific primary antibodies overnight, and either horseradish peroxidase-conjugated goat anti-rabbit or anti-mouse antibodies for 2 h. The blots are detected using VL Chemi-Smart 3000 (Viogene Biotek, Sunnyvale, Calif.) with the enhanced chemiluminescence (ECL) western blotting reagent (Millipore, Billerica, Mass.).

J. Immunoprecipitation

HNDFs are seeded at a density of 1×10⁶ cells/dish in 10 cm dish and treated with ANM or pharmacological inhibitors of AKT, JNK/SAPK, p38 MAPK and ERK1/2 in the presence or absence of HG. After treatment, cells are lysed with RIPA buffer containing protease inhibitor cocktail. The lysates are homogenized and centrifuged at 16,000×g for 15 min at 4° C. The supernatant is collected and the protein concentration is determined by Bio-Rad protein assay reagent. Total protein extract containing 500 μg of proteins are precleared with protein-A agarose beads for 1 h and incubated with 3 μg of anti-Keap-1 antibody for overnight at 4° C. with gently shake. After overnight incubation, centrifuged at 2000×g for 5 min at 4° C., the supernatant is discarded and the reaming pellet is washed with RIPA buffer. Immunoprecipitated complexes are mixed with SDS sample buffer and denatured at 94° C. for 5 min. Equal amount of protein samples are subjected to western blotting. The ubiquitinated Keap-1 protein levels are determined by ubiquitin antibody.

K. Gene Silencing by siRNA

HNDFs (2.5×10⁵ cells/dish) are cultured in 6 cm dishes, after 60% confluence at the time of transfection, culture media is replaced with 2 mL of Opti-MEM (Invitrogen) and cells are transfected using Lipofectamine RNAiMax (Invitrogen) transfection reagent. For each transfection, 5 μL of RNAiMAX is mixed with 500 μL of Opti-MEM and incubated for 5 min at room temperature. In a separate tube, siRNA (100 μM for a final concentration of 100 nM in 1 mL Opti-MEM) is added to 500 μL of Opti-MEM and the siRNA solution is added to the diluted RNAiMAX reagent. The resulting siRNA/RNAiMAX mixture (1 mL) is incubated for an additional 25 min at room temperature to allow complex formation. Subsequently, the solution is added to the cells in the 6-well plates, giving a final transfection volume of 2 mL. After 6 h incubation, the transfection medium is replaced with 3 mL of standard growth medium and the cells are cultured at 37° C. After transfection for 24 h, cells are treated with ANM, NAC or RES in the presence or absence of HG, and subjected to subsequent experiments.

L. Luciferase Reporter Assay

ARE promoter activity is measured using a dual-luciferase reporter assay system (Promega, Madison, Wis.). Briefly, HNDFs or HUVECs (1×10⁵ cells/well) are cultured in 6-well plates until ˜80% confluence and then incubated for 5 h in Opti-MEM that did not contain antibiotics. Cells are then transfected with ARE plasmid (Qiagen, Hilden, Germany) using Lipofectamine 2000 (Invitrogen) and incubated for 36 h. After plasmid transfection, cells are treated with ANM (10 μM) or NAC (100 μM) or RES (5 μM) in the presence or absence of HG (30 mM) for 6 h. The cell lysate is prepared and incubated with luciferase agents and the relative luminescence intensity is quantified using a spectrophotometer (Hidex Oy, Turku, Finland).

M. C. elegans Strain

The wild type Bristol N2 strain is used in the present invention. C. elegans and Escherichia coli OP50 strain are obtained from the Caenorhabditis Genetic Center, University of Minnesota (Minneapolis-St. Paul, Minn.). Worms are maintained at 20° C. on nematode growth medium (NGM). Hatched worms (L1 -stage larvae) are transferred to fresh agar plates and cultured with Escherichia coli (E. coli) OP50 as a food source until they reached the L4 larvae stage. Synchronization of worm cultures is achieved by hypochlorite treatment of gravid hermaphrodites.

N. Stress-Resistance Assay

Age synchronized L1 larvae are incubated with liquid S-basal medium containing E. coli OP50 at a density of 1×10⁹ cells. MI and 10 and 20 μM ANM or 0.01% DMSO (vehicle control) for 3 days. Subsequently, adult worms are subjected to oxidative stress assay. To induce oxidative stress, worms are incubated with Juglone (5-hydroxyl-1,4-naphthoquinone; Sigma), an ROS-generating agent. ANM treated and control worms are transferred to S-basal medium containing 250 μM Juglone, and incubated from 2.5, 3.5 and 4.5 h. After treatment, viable worms are scored. Worms are scored as dead when they failed to response physical touch. The test is performed triplicate.

O. Hyperglycemia-Induced Lifespan Assay

For the lifespan assay, age synchronized L1 larvae are transferred to NGM plates containing ANM (10 μM) or RES (438 μM) with or without high-glucose (50 mM). Control worms are treated with 0.01% DMSO. All worms are kept 20° C. to develop adulthood. After 6 days, worms are transferred to plates containing glucose are applicable. Surviving and dead animals are counted daily (starting from the first day of adulthood) until all worms had died. Animals that did not move when gently prodded are scored as dead. Worms suffering from internal hatch (a defect in egg-laying) and those that crawled off the NGM plate are not included in the life-span assay. During the reproductive period, adult worms are transferred to fresh NGM plates every day during the progeny production period and then every other day thereafter. Life span assay result is obtained from three independent assays.

P. Statistical Data Analysis

Data are expressed as mean±S.E.M. All data are analyzed using the statistical software Graphpad Prism version 6.0 for windows (Graph Pad Software, La Jolla, Calif.). Statistical analysis is performed using one-way ANOVA followed by Dunnett's multiple comparisons test with a P value of less than 0.05 indicating statistical significance.

The present invention reveals the anti-aging properties of ANM (ANM) and elucidates the molecular mechanism underlying the effects. It is found that exposure of HNDFs to high-glucose (HG, 30 mM) for 3 days, G0/G1 phase arrest and senescence are accelerated. Indeed, co-treatment with ANM (10 μM) significantly attenuates HG-induced growth arrest and promotes cell proliferation. Further molecular analysis reveals that ANM blocks the HG-induced reduction in G1-S transition regulatory proteins such as cyclin D, cyclin E, CDK4, CDK6, CDK2 and protein retinoblastoma (pRb). In addition, treatment with ANM eliminates HG-induced reactive oxygen species (ROS) through the induction of anti-oxidant genes, HO-1 and NQO-1 via transcriptional activation of Nrf2. Moreover, treatment with ANM abolishes HG-induced SIPS as evidenced by reduced senescence-associated β-galactosidase (SA-β-gal) activity. This effect is further confirmed by reduction in senescence-associated marker proteins including, p21^{CIP1}, p16^{INK4A}, and p53/FoxO1 acetylation. Also, the HG-induced decline in aging-related marker protein SMP30 is rescued by ANM. Furthermore, treatment with ANM increases SIRT-1 expression, and prevents SIRT-1 depletion. This protection is consistent with inhibition of SIRT-1 phosphorylation at Ser47 followed by blocking its upstream kinases, p38 MAPK and JNK/SAPK. Further analysis reveals that ANM partially protects HG-induced senescence in SIRT-1 silenced cells. A similar effect is also observed in Nrf2 silenced cells. However, a complete loss of protection is observed in both Nrf2 and SIRT-1 knockdown cells suggesting that both induction of Nrf2-mediated anti-oxidant defense and SIRT-1-mediated deacetylation activity contribute to the anti-aging properties of ANM in vitro. Result of in vivo studies shows that ANM-treated C. elegens exhibits an increased survival rate during HG-induced oxidative stress insult. Furthermore, ANM significantly extends the life span of C. elegans. Taken together, the present invention suggests the potential application of ANM in age-related diseases or as a preventive reagent against aging process.

To sum up, the present invention is a pharmaceutical composition having anti-aging properties against high-glucose, where an anti-aging reagent of a steroid-like phytochemical ANM eliminates hyperglycemia-accelerated premature senescence in HNDFs by direct activation of Nrf2 and SIRT-1.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

### Abbreviations:

| | |
|---|---|
| ANA | antcin A |
| ANB | antcin B |
| ANC | antcin C |
| ANH | antcin H |
| ANK | antcin K |
| ANM | antcin M |
| CDK | cyclin-dependent kinase |
| DAPI | 4′,6-diamidino-2-phenylindole |
| DCFH-DA | 2′,7′-dichlorofluorescein diacetate |
| FBS | fetal bovine serum |
| HG | high-glucose |
| HNDFs | human normal dermal fibroblasts |
| HO-1 | hemeoxygenase-1 |
| HUVECs | human umbilical vein endothelial cells |
| MEM | minimum essential medium |
| MTT | 3-(4,5-dimethyl-thiazol-2-yl)-2,5-diphenyl |
| | tetrazolium bromide |
| NAC | N-acetylcysteine |
| NG | normal-glucose |
| NQO-1 | NAD(P)H:quinone oxidoreductase 1 |
| Nrf2 | NF-E2 related factor-2 |
| PRb | protein retinoblastoma |
| RES | resveratrol |
| ROS | reactive oxygen species |
| SA-β-gal | senescence-associated β-galactosidase |
| SIPS | stress-induced premature senescence |
| SIRT-1 | silent mating type information regulation 2 |
| | homologs 1 |
| SMP30 | senescence marker protein-30 |

1. A method of treating aging of cells, comprising administering to a subject an effective amount of a pharmaceutical composition comprising antcin M (ANM) isolated from Antrodia salmonea as an active ingredient, and a pharmaceutically acceptable carrier or excipient, wherein said antcin M is isolated to remove cytotoxic concentrations of antcin B and antcin K and wherein the cells are dermal fibroblasts.

2. The method according to claim 1, wherein the cells are human normal dermal fibroblasts (HNDFs).

3. The method according to claim 2, wherein the composition protects HNDFs to prevent hyperglycemia-induced G₀/G₁ phase arrest and senescence.

4. The method according to claim 1, wherein the composition inhibits high-glucose (HG) -induced reduction in G1-S transition regulatory proteins.

5. The method according to claim 4, wherein said G1-S transition regulatory proteins comprises cyclin D, cyclin E, cyclin-dependent kinase (CDK4), CDK6, CDK2 and protein retinoblastoma (pRb).

6. The method according to claim 1, wherein the composition protects HNDFs to prevent hyperglycemia-induced oxidative damage.

7. The method according to claim 1, wherein the composition activates NF-E2 related factor-2 (Nrf2) -mediated anti-oxidant genes and eliminates HG-induced reactive oxygen species (ROS).

8. The method according to claim 7, wherein said anti-oxidant genes comprises hemoxygenase-1 (HO-1) and NAD(P)H:quinone oxidoreductase-1 (NQO-1).

9. The method according to claim 1, wherein the composition abolishes stress-induced premature senescence (SIPS) in the presence of HG by reducing senescence-associated β-galactosidase (SA-β-gal) activity in HNDFs.

10. The method according to claim 1, wherein the composition reduces expression of senescence-associated marker proteins in HNDFs, selected from the group consisting of: p21^{CIP1}, p16^{INK4A}, and p53/FoxO1 acetylation.

11. The method according to claim 1, wherein the composition increases expression of silent mating type information regulation 2 homologs 1 (SIRT-1) in HNDFs.

12. The method according to claim 1, wherein the composition enhances expression of senescence marker protein-30 (SMP30) in HG-induced HNDFs.

13. The method according to claim 1, wherein the composition protects and extends the life span of Caenorhabditis elegans (C. elegans) under stress conditions.

14. The method of claim 1, wherein said pharmaceutical composition is administered at a concentration from 10 μM to 30 μM.

15. The method of claim 1, wherein said pharmaceutical composition further comprises ANA, ANH or ANC at a non-cytotoxic concentration.

16. The method of claim 1, wherein the pharmaceutical composition is applied topically.

17. The method of claim 1, wherein the method is used to treat the skin symptoms of diabetes.

18. The method of claim 1, wherein ANM is isolated from the fruiting bodies of A. salmonea.

19. The method of claim 1, wherein the purity of the ANM is 99% as confirmed by HPLC and FT-NMR analysis.

20. A method of reducing the senescence of dermal fibroblast cells, comprising administering to a subject an effective amount of a pharmaceutical composition, comprising antcin M (ANM) isolated from Antrodia salmonea as an active ingredient, and a pharmaceutically acceptable carrier or excipient, wherein said antcin M is 99% pure and wherein said antcin M is isolated to remove cytotoxic concentrations of antcin B and antcin K.

21. The method of claim 1, wherein the pharmaceutical composition further comprises resveratrol or N-acetyl cysteine (NAC).
